(19) United States
(12) Patent Application Publication
(10) Pub. No.: US 2018/0125975 A1
(43) Pub. Date: May 10, 2018

(54) EMISSIVE POLYMERIC MATRICES

(71) Applicant: KLOX Technologies Inc., Laval (CA)

(72) Inventors: Remigio Piergallini, Grottammare Ascoli Piceno (IT); Nikolaos Loupis, Athens (GR); Liviu Dragomir, Saint-Leonard (CA)

(73) Assignee: KLOX Technologies Inc., Laval (CA)

(21) Appl. No.: 15/511,088

(22) PCT Filed: Sep. 15, 2015
(86) PCT No.: PCT/CA2015/050890

§ 371 Date: Mar. 14, 2017

Related U.S. Application Data
(60) Provisional application No. 62/050,685, filed on Sep. 15, 2014.

(51) Int. Cl. A61K 41/00; A61K 47/32; A61K 8/81; A61K 9/06; A61K 8/04; A61K 47/14; A61K 8/37; A61K 47/08; A61K 8/38; A61K 31/352; A61K 8/49; A61K 47/10; A61K 8/34; A61K 9/00; A61Q 19/08; A61N 5/06

(52) U.S. Cl. CPC A61K 41/0052; A61N 2005/067; A61K 8/8152; A61K 9/06; A61K 8/042; A61K 47/14; A61K 8/37; A61K 47/08; A61K 8/38; A61K 31/352; A61K 8/498; A61K 47/10; A61K 8/345; A61K 9/0014; A61Q 19/08; A61N 5/0616; A61N 5/062; A61K 2800/434; A61K 2800/81; A61N 2005/0651; A61N 2005/0654; A61K 47/32

(57) ABSTRACT

The present disclosure relates to emissive polymeric matrices and methods of using them. The emissive polymeric matrices comprise a monomer such as, for example, 2-hydroxyehtyl methacrylate (HEMA) and at least one chromophore.

### CROSS-REFERENCE TO RELATED APPLICATIONS

This application claims the benefit of and priority to U.S. provisional patent application No. 62/050,685, filed Sep. 15, 2014, the content of which is herein incorporated in its entirety by reference.

### FIELD OF TECHNOLOGY

The present disclosure generally relates to emissive polymeric matrices, to methods of forming them and to the potential uses thereof.

### BACKGROUND OF THE DISCLOSURE

Phototherapy has been recognized as having a wide range of applications in both the medical and cosmetic fields including use in surgery, therapy and diagnostics. For example, phototherapy has been used to treat cancers and tumors with lessened invasiveness, to disinfect target sites as an antimicrobial treatment, to treat skin conditions, to promote wound healing, and for facial skin rejuvenation.

Up to now, phototherapy has been achieved using mainly liquid formulations which are to be applied onto a surface to be treated. Some drawbacks of liquid formulations are that, due to their liquid consistency, they tend to spread beyond the surface to be treated and/or tend to get diluted when they are in contact with other liquids (such as water) or semi-liquids.

Hydrogels have been proposed as a matrix for use in a number of applications, including surgery, medical diagnosis and treatment, adhesives and sealers. Because of their flexible structure, hydrogels present some advantages for medical applications. However, these hydrogels have been shown to absorb solvents (such as water) and undergo swelling and lose three-dimensional network ability of reversible deformation.

It is thus an object of the present disclosure to provide new and improved matrices that may solve at least some of the drawbacks observed to date that may be used in phototherapy and that may have industrial applicability.

### SUMMARY OF THE DISCLOSURE

According to various aspects, the present disclosure relates to emissive polymeric matrices useful in phototherapy. According to various aspects, the present disclosure relates to emissive polymeric matrices useful in phototherapy and in biophotonic therapy and/or industrial applicability.

In particular, emissive polymeric matrices of the present disclosure include a polymerisable monomer, and at least one chromophore. Preferably, the at least one chromophore can absorb and/or emit light.

In some instances, the emissive polymeric matrices are substantially resistant to leaching, such as, when being in storage such that less than about 10%, or less than about 9%, or less than about 8%, or less than about 7%, or less than about 6%, or less than about 5%, or less than about 4%, or less than about 3%, or less than about 2%, or less than about 1%, or less than about 0.5%, or less than about 0.1% by weight of the total chromophore amount leaches out of the matrix. Leaching of the chromophore may be measured by (i) placing a 2 mm thick emissive polymeric matrix onto a top surface of a 2.4-3 cm diameter polycarbonate (PC) membrane with a thickness of 10 microns and a pore size of 3 microns, (ii) contacting a bottom surface of the PC membrane with a phosphate saline buffer solution contained in a receptor compartment, and (iii) after a treatment time at room temperature and pressure, measuring the chromophore content in the receptor compartment.

In some implementations, the polymerisable monomer is 2-hydroxyethyl methacrylate (HEMA).

In some implementations, the chromophore of the present hydrogel composition may be a xanthene dye. The xanthene dye may be Fluorescein or eosin, or any other xanthene dye. In some instances, the at least one chromophore is Eosin Y. In some other instances, the chromophore is Fluorescein. In some other instances, the chromophores are Eosin Y and Fluorescein. In some instances, the chromophore is Rose Bengal. In some other instances, the chromophore is Eosin Y and Rose Bengal. In some other instances, the chromophore is Eosin Y, Rose Bengal and Fluorescein.

In some embodiments, the emissive polymeric matrix further comprises a cross-linker. In some implementations, the cross-linker is ethylene glycol dimethacrylate (EGDMA). The composition may also include an initiator. In some implementations, the initiator is benzoyl peroxide (BPO).

In some embodiments, the chromophore absorbs and/or emits light within the range of about 400 nm and about 750 nm or about 400 nm and about 700 nm.

The emissive polymeric matrices of the present disclosure may be used for medical treatment of a tissue. The cosmetic treatment may be skin rejuvenation and conditioning, treatment of wound healing, treatment of acne or of other skin conditions including acne, eczema, psoriasis or dermatitis. In some implementations, the tissue is from a human or from an animal.

In some aspects, the animal is for example, a cat, dog, horse, sheep, goat, cow, pig, hamster, guinea pig, or a rabbit. The emissive polymeric matrix can be used to promote treatment of the skin of an animal and/or treat animal such as, but not limited to, constant scratching, licking and chewing at the skin, scabs, redness or inflammation, round, scaly patches on the face and paws, dryness, flaky, irritated skin, rashes, swellings, lumps or skin discoloration, drainage of blood or pus. They can be used to treat infections or acute inflammation in animals. Acute inflammation in animals can present itself as pain, heat, redness, swelling and loss of function, and includes inflammatory responses such as those seen in allergic reactions such as those to insect bites e.g.; mosquito, bees, wasps, poison ivy, or post-ablative treatment.

In some aspects, the emissive polymeric matrices of the present disclosure may be used for modulating inflammation, modulating collagen synthesis or for promoting angiogenesis. In some implementations of these aspects, the emissive polymeric matrices of the present disclosure may be used for modulating the production of pro-inflammatory cytokines and for improving the healing process.

The present disclosure also provides methods for promoting wound healing comprising applying an emissive polymeric matrix according to the present disclosure over a wound, wherein the emissive polymeric matrix comprises 2-Hydroxyethyl methacrylate (HEMA) and at least one chromophore.

The present disclosure also provides methods for promoting wound healing comprising applying an emissive polymeric matrix according to the present disclosure over a wound, wherein the emissive polymeric matrix comprises 2-hydroxyethyl methacrylate (HEMA) and at least one chromophore; and illuminating said matrix with light having a wavelength that is absorbed by the at least one chromophore, wherein said method promotes wound healing.

The present disclosure also provides emissive polymeric matrices and methods for preventing or treating infections comprising applying a emissive polymeric matrix on a target skin tissue, wherein the emissive polymeric matrix comprises 2-hydroxyethyl methacrylate (HEMA), and at least one chromophore; and illuminating said emissive polymeric matrix with light having a wavelength that is absorbed the at least one chromophore; and wherein said method treats the infected tissue or prevents the tissue from becoming infected. In some embodiments, the infection is a bacterial infection, a fungal infection or a viral infection.

The present disclosure also provides methods for treating a skin disorder, wherein the method comprises applying an emissive polymeric matrix over a target skin tissue, wherein the emissive polymeric matrix comprises 2-hydroxyethyl methacrylate (HEMA), and at least one chromophore; and illuminating said emissive polymeric matrix with light having a wavelength that is absorbed by the at least one chromophore; and wherein said method promotes healing of said skin disorder. In some embodiments, the skin disorder is selected from acne, eczema, psoriasis and dermatitis.

The present disclosure also provides methods for treating acne comprising: applying an emissive polymeric matrix over a target skin tissue, wherein the emissive polymeric matrix comprises 2-hydroxyethyl methacrylate (HEMA), and at least one chromophore; and illuminating said emissive polymeric matrix with light having a wavelength that is absorbed by the at least one chromophore; and wherein said method treats the acne.

The present disclosure also provides methods for skin rejuvenation comprising applying an emissive polymeric matrix over a target skin tissue, wherein the emissive polymeric matrix comprises 2-hydroxyethyl methacrylate (HEMA), and at least one chromophore; and illuminating said emissive polymeric matrix with light having a wavelength that is absorbed by the at least one chromophore; and wherein said method promotes skin rejuvenation.

The present disclosure also provides methods for preventing or treating scars comprising applying a emissive polymeric matrix on a target skin tissue, wherein the emissive polymeric matrix comprises 2-hydroxyethyl methacrylate (HEMA), and at least one chromophore; and illuminating said emissive polymeric matrix with light having a wavelength that is absorbed the at least one chromophore; and wherein said method promotes wound healing.

The present disclosure also provides emissive polymeric matrices and methods for preventing or treating a bone diseases or conditions.

The present disclosure also provides emissive polymeric matrices and methods for preventing or treating an oral diseases or conditions.

The present disclosure also provides emissive polymeric matrices and methods for preventing or treating orphan diseases.

In some implementations of the methods defined herein, the emissive polymeric matrices are in direct contact with the area of the tissue in need of phototherapy. That is to say that the emissive polymeric matrix touches at least partially the area to be treated.

In some implementations of the methods defined herein, the emissive polymeric matrices are not in direct contact with the area of the tissue in need of phototherapy. That is to say that the emissive polymeric matrix does not touch the area to be treated. In some instances, the emissive polymeric matrix may be less than 1 cm from the area to be treated, or may be less than 2 cm, or less than 3 cm, or less than 4 cm, or less than 5 cm, or less than 10 cm from the area to be treated.

According to various aspects, the present disclosure relates to an emissive polymeric matrix comprising: 2-hydroxyehtyl methacrylate (HEMA), and at least one chromophore.

According to various aspects, the present disclosure relates to a method for promoting wound healing comprising: placing an emissive polymeric matrix over a wound, wherein the matrix comprises 2-hydroxyehtyl methacrylate (HEMA) and at least one chromophore; and illuminating said matrix with light having a wavelength that is absorbed by the at least one chromophore; wherein said method promotes wound healing.

According to various aspects, the present disclosure relates to a method for biophotonic treatment of a skin disorder comprising: placing an emissive polymeric matrix over a target skin tissue, wherein the matrix comprises 2-hydroxyehtyl methacrylate (HEMA) and at least one chromophore; and illuminating said matrix with light having a wavelength that is absorbed by the at least one chromophore; and wherein said method promotes healing of said skin disorder.

According to various aspects, the present disclosure relates to a method for biophotonic treatment of acne comprising: placing an emissive polymeric matrix over a target skin tissue, wherein the matrix comprises 2-hydroxyehtyl methacrylate (HEMA), and at least one chromophore; and illuminating said matrix with light having a wavelength that is absorbed by the at least one chromophore; and wherein said method treats the acne.

According to various aspects, the present disclosure relates to a method for promoting skin rejuvenation comprising: placing an emissive polymeric matrix over a target skin tissue, wherein the matrix comprises 2-hydroxyehtyl methacrylate (HEMA), and at least one chromophore; and illuminating said matrix with light having a wavelength that is absorbed by the at least one chromophore; and wherein said method promotes skin rejuvenation.

According to various aspects, the present disclosure relates to a method for preventing or treating scars comprising: placing an emissive polymeric matrix a target skin tissue, wherein the matrix comprises 2-hydroxyehtyl methacrylate (HEMA), and at least one chromophore; and illuminating said matrix with light having a wavelength that is absorbed the at least one chromophore; and wherein said method promotes wound healing.

According to various aspects, the present disclosure relates to a method for biophotonic skin treatment comprising: placing an emissive polymeric matrix over a skin, wherein the matrix comprises 2-hydroxyehtyl methacrylate (HEMA) and at least one chromophore; and illuminating said matrix with light having a wavelength that is absorbed by the at least one chromophore; and wherein said method promotes treatment of said skin.

According to some embodiments, the emissive polymeric matrices of the present disclosure may generate radiation when exposed to light, said radiation being capable of inducing thermal effects on the surrounding environment of the emissive polymeric matrices. In some aspects, the emissive polymeric matrices of the present disclosure may be used for generating thermal radiation. In some aspects, the emissive polymeric matrices of the present disclosure may be used for generating infrared.

### BRIEF DESCRIPTION OF THE DRAWINGS

Further aspects of the present disclosure will become better understood with reference to the description in association with the following in which:

FIGS. 1A to 1C: FIG. 1A illustrates the light emission spectra of a 4 mm thick pHEMA transparent disk comprising 50 mg Eosin and 50 mg Fluorescein (E50+F50) according to an embodiment of the present disclosure during a 5 minute illumination period. FIG. 1B illustrates the light emission spectra of a 2.0 mm thick pHEMA transparent disc comprising 100 mg Fluorescein (F100) according to an embodiment of the present disclosure during a 5 minute illumination period. FIG. 1C illustrates the light emission spectra of a 2.3 mm thick pHEMA disk comprising 50 mg Eosin and 50 mg Fluorescein (E50+F50) according to an embodiment of the present disclosure during a 5 minute illumination period according to an embodiment of the present disclosure.

FIG. 2 illustrates the light emission spectra of a 2.3 mm thick block opaque pHEMA matrix comprising 50 mg Eosin and 50 mg Fluorescein (E50+F50) according to an embodiment of the present disclosure during a 5 minute illumination period.

FIG. 3 illustrates the light emission spectra of a 2.3 mm thick pHEMA hydrogel comprising 50 mg Fluorescein (F50) according to an embodiment of the present disclosure during a 5 minute illumination period.

FIG. 4 illustrates the light emission spectra of a 2.0 mm thick silicone, pHEMA microspheres comprising 100 mg Eosin (E100) according to an embodiment of the present disclosure during a 5 minute illumination period.

FIG. 5 illustrates a graph showing IL8 secretion by IFNγ stimulated HaCaT cells 24 hours after treatment with the indicated emissive polymeric matrices and indicated light for 5 minutes at a distance of 5 mm.

FIG. 6 illustrates a graph showing IL6 secretion by IFNγ stimulated HaCaT cells 24 hours after treatment with the indicated emissive polymeric matrices and indicated light for 5 minutes at a distance of 5 mm.

FIG. 7 illustrates a graph showing IL6 secretion by HaCaT keratinocytes 24 hours after treatment with light from a blue light and the indicated emissive polymeric matrices according to one embodiment of the present disclosure.

FIG. 8 illustrates a graph showing IL8 secretion by HaCaT keratinocytes 24 hours after treatment with light from a blue light and the indicated emissive polymeric matrices according to one embodiment of the present disclosure.

FIGS. 9A, 9B and 9C illustrate graphs showing the temperature of water placed in a container made of a pHEMA matrix according to one embodiment of the present disclosure.

FIG. 10 illustrates a picture of an experimental set-up according to one aspect of the present disclosure.

FIGS. 11A and 11B: FIG. 11A illustrates a schematic representation of an experimental protocol designed to determine the effect of the emissive polymeric matrices of the present disclosure on wound healing using a 3D human skin model. (A) denotes the wounded area and (B) denotes the unwounded area. FIG. 11B illustrates a further schematic representation of the experimental protocol of FIG. 11A.

FIGS. 12A-12G: FIG. 12A illustrates a histochemical view of full excision (A) of 3D human skin model at day 0. FIG. 12B illustrates a histochemical view of full excision (A) of 3D human skin model 1 day after the excision wherein the skin has been kept in the dark (CTRL=control). FIG. 12C illustrates a histochemical view of full excision of 3D human skin model 1 day after the excision wherein the skin has been treated for 5 minutes with ambient light (LIGHT=ambient light). FIG. 12D illustrates a histochemical view of full excision of 3D human skin model 1 day after the excision wherein the skin has been treated for 5 min using a 450 nm light passing through a pHEMA matrix (pHEMA)). FIG. 12E illustrates a histochemical view of full excision of 3D human skin model 3 days after the excision wherein the skin has been kept in the dark (CTRL=control). FIG. 12F illustrates a histochemical view of full excision of 3D human skin model 3 days after the excision wherein the skin has been treated for 5 minutes with ambient light (LIGHT=ambient light). FIG. 12G illustrates a histochemical view of full excision of 3D human skin model 3 days after the excision wherein the skin has been treated for 5 min using a 450 nm light passing through a pHEMA matrix (pHEMA)).

FIG. 13 illustrates a graph indicating the relationship between the thickness of the epidermis versus the development of the stratum corneum 3 days post treatment with the emissive polymeric matrix of the present disclosure in a 3D human skin model.

FIG. 14 illustrates a picture showing the elastic properties of an emissive polymeric matrix according to one embodiment of the present disclosure.

### DETAILED DESCRIPTION

The present disclosure relates to emissive polymeric matrices and uses thereof.

In various aspects, the present disclosure relates to the use of emissive polymeric matrices defined herein for biophotonic therapy. Biophotonic therapy would provide the beneficial effects of inducing photobiostimulation by the fluorescent light generated upon illumination of the materials forming the emissive polymeric matrices.

In some implementations of these aspects, the biophotonic therapy may include photothermal therapy which may be achieved using these emissive polymeric matrices. Photothermal therapy would provide the beneficial effects of inducing photobiostimulation by the radiant energy of the electromagnetic radiation generated upon illumination of the materials forming the matrices.

In certain embodiments, the emissive polymeric matrices of the present disclosure are activated by visible light. Furthermore, in certain embodiments, biophotonic therapy using the emissive polymeric matrices of the present disclosure may for instance promote wound healing, rejuvenate the skin by, e.g., promoting collagen synthesis, treat skin conditions such as acne, and treat periodontitis.

In some implementations of these embodiments, the emissive polymeric matrices of the present disclosure are in a solid form. In some other implementations, the emissive polymeric matrices of the present disclosure are in a semi-solid form. As used herein, the expression “semi-solid” refers to something that lies along the boundary between a solid and a liquid. While similar to a solid in some respects, in that semi-solids can support their own weight and hold their shapes, a semi-solid also shares some properties of liquids, such as conforming in shape to something applying pressure to it and the ability to flow under pressure. In some instance, the emissive polymeric matrices have a water content that is between about 20% and 40% of the weight of the emissive polymeric matrix, preferably between about 25% and 35%, more preferably about 30%.

The emissive polymeric matrices of the present disclosure may be shaped or molded into any convenient shape and/or form. As such, the emissive polymeric matrices defined herein are not limited to any particular geometric shape. In some instances, the emissive polymeric matrix of the present disclosure may be shaped into an article of clothing such as, but not limited to a glove, or into a bandage.

In some other implementations, the emissive polymeric matrices may be shaped into an article useful in a medical treatment or into an article useful in dental treatment.

The shape of the emissive polymeric matrices may be irregular so as to create physical attachment points or locations to assist with retention of the emissive polymeric matrices into or onto a substrate such as, but not limited to, a fabric, a textile, fibers, foams or the like. The surface of the emissive polymeric matrices or parts thereof may be irregular, discontinuous and/or rough. The surface of the emissive polymeric matrices or parts thereof may also be regular, continuous and/or smooth.

In some implementations, the emissive polymeric matrices of the present disclosure are elastic. As used herein, the term “elastic” refers to ability of an emissive polymeric matrix of the present disclosure to resist a distorting influence or stress and to return to its original size and shape when the stress is removed. If the emissive polymeric matrix is elastic, the matrix will return to its initial shape and size when these forces are removed. The emissive polymeric matrices may be made elastic by, for example, varying the concentration of certain materials entering into the composition of the emissive polymeric matrices.

In some implementations, the emissive polymeric matrices of the present disclosure may be shaped into a powder.

In some implementations, the polymeric matrices of the present disclosure may be shaped into particles such as microparticles and/or microspheres or such as nanoparticles and/or nanospheres. The particles of the present disclosure may be applied into or into a substrate. Preferably, the substrate is porous. A porous substrate refers to a substrate that has interstices, recesses, pores or cross linkage openings in which the microspheres can be retained, impregnated or trapped. The interstices, recesses or cross linkage openings are preferably of a dimension that permits insertion and/or retention of the particles.

It is to be understood that this disclosure is not limited to specific compositions or process steps, as such may vary. It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 15%, preferably within 14%, preferably within 13%, preferably within 12%, preferably within 11%, preferably within 10%, preferably within 9%, preferably within 8%, preferably within 7%, preferably within 6%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the expression “emissive polymeric matrices” refers to matrices comprising polymers and having the ability to emit electromagnetic radiation such as, for example but not limited to, light, or infrared.

As used herein, the expression “emissive polymeric matrix composition” refers to compositions which upon polymerization results in the emissive polymeric matrix as defined herein having a solid or a semi-solid form. In some instance, the emissive polymeric matrix composition comprises monomers that have not been polymerized or that are in a substantially non-polymerized state.

“Biophotonic” refers to the generation, manipulation, detection and application of photons in a biologically relevant context. In other words, biophotonic compositions and materials exert their physiological effects primarily due to the generation and manipulation of photons.

“Photothermal therapy” refers to use of electromagnetic radiation (e.g., infrared) for the treatment of medical conditions.

“Hydrogel” refers to a material of solid or semi-solid texture that includes water. Hydrogels are formed by a three-dimensional network of molecular structures within which water, among other substances, may be held. The three-dimensional molecular network may be held together by covalent chemical bonds, or by ionic bonds, or by any combination thereof. Some hydrogels may be formed through the mixture of two or more materials that undergo chemical or physical reactions with each other to create the three-dimensional molecular network that provides the hydrogel with a degree of dimensional stability.

By “in use” is meant during a treatment time which can be up to about 5 minutes, up to about 6 minutes, up to about 7 minutes, up to about 8 minutes, up to about 9 minutes, up to about 10 minutes, up to about 15 minutes, up to about 20 minutes, up to about 25 minutes, or up to about 30 minutes. The treatment time may comprise the total length of time that the emissive polymeric matrix is either in contact with a surface to be treated or in proximity of a surface to be treated (i.e., without being in direct contact with the surface to be treated).

In some implementations, the surface to be treated is, for example, but not limited to, skin, mucous membranes, vagina, oral cavity, internal surgical wound sites, and the like.

“Topical application” or “topical uses” means application to body surfaces, such as the skin, mucous membranes, vagina, oral cavity, internal surgical wound sites, and the like.

The term “chromophore” is used herein means a chemical compound, when contacted by light irradiation, is capable of absorbing the light. The chromophore readily undergoes photoexcitation and can transfer its energy to other molecules or emit it as light (fluorescence).

“Photobleaching” or “photobleaches” means the photochemical destruction of a chromophore. A chromophore may fully or partially photobleach.

The term “actinic light” is intended to mean light energy emitted from a specific light source (e.g. lamp, LED, or laser) and capable of being absorbed by matter (e.g. the chromophore). Terms “actinic light” and “light” are used herein interchangeably. In a preferred embodiment, the actinic light is visible light.

“Skin rejuvenation” means a process of reducing, diminishing, retarding or reversing one or more signs of skin aging or generally improving the condition of skin. For instance, skin rejuvenation may include increasing luminosity of the skin, reducing pore size, reducing fine lines or wrinkles, improving thin and transparent skin, improving firmness, improving sagging skin (such as that produced by bone loss), improving dry skin (which might itch), reducing or reversing freckles, reducing or preventing the appearance of age spots, spider veins, rough and leathery skin, fine wrinkles that disappear when stretched, reducing loose skin, or improving a blotchy complexion. According to the present disclosure, one or more of the above conditions may be improved or one or more signs of aging may be reduced, diminished, retarded or even reversed by certain embodiments of the compositions, methods and uses of the present disclosure.

“Wound” means an injury to any tissue, including for example, acute, subacute, delayed or difficult to heal wounds, and chronic wounds. Examples of wounds may include both open and closed wounds. Wounds include, for example, amputations, burns, incisions, excisions, lesions, lacerations, abrasions, puncture or penetrating wounds, surgical wounds, amputations, contusions, hematomas, crushing injuries, ulcers (such as for example pressure, diabetic, venous or arterial), scarring (cosmesis), and wounds caused by periodontitis (inflammation of the periodontium).

Features and advantages of the subject matter hereof will become more apparent in light of the following description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature and not as restrictive and the full scope of the subject matter is set forth in the claims.

Emissive Polymeric Matrices

The present disclosure provides emissive polymeric matrices and methods of using the emissive polymeric matrices. In some implementations, the emissive polymeric matrices are activated by light (e.g., photons) of specific wavelength. When an emissive polymeric matrix is said to be activated, it indicates that the components of the matrix have been activated by light and are capable of emitting/radiating, for example, light and/or heat.

Emissive polymeric matrices according to various embodiments of the present disclosure comprise at least one polymerisable monomer and at least one chromophore. Illumination of the emissive polymeric matrices can activate the at least one chromophore. When a chromophore absorbs a photon of a certain wavelength, it becomes excited. This is an unstable condition and the molecule tries to return to the ground state, giving away the excess energy. For some chromophores, it is favorable to emit the excess energy as light when returning to the ground state. This process is called fluorescence. The peak wavelength of the emitted fluorescence is shifted towards longer wavelengths compared to the absorption wavelengths due to loss of energy in the conversion process. This is called the Stokes' shift.

In the proper environment (e.g., in an emissive polymeric matrix) much of this energy is transferred to the other components of the matrix and/or to the treatment site directly; and/or to the environment surrounding the matrix.

Without being bound to theory, it is thought that fluorescent light emitted by photoactivated chromophores may have therapeutic properties due to its femto-, pico-, or nano-second emission properties which may be recognized by biological cells and tissues, leading to favourable biomodulation. Furthermore, the emitted fluorescent light has a longer wavelength and hence a deeper penetration into the tissue than the activating light. Irradiating tissue with such a broad range of wavelength, including in some embodiments the activating light which passes through the composition, may have different and complementary effects on the cells and tissues. In other words, chromophores may be used in the emissive polymeric matrices of the present disclosure for therapeutic effect on tissues.

In some implementations, the emissive polymeric matrices of the present disclosure may have topical uses such as a mask or a wound dressing. In some instances, the emissive polymeric matrices may have a topic effect without being in direct contact with the skin. In such instances, the emissive polymeric matrices may be used as, for example, an attachment to a light source, as a waveguide or as a light filter. In addition the emissive polymeric matrices can limit the contact between the chromophore and the tissue.

These materials may be described based on the components making up the composition. Additionally or alternatively, the compositions of the present disclosure have functional and structural properties and these properties may also be used to define and describe the compositions. Individual components of the emissive polymeric matrices of the present disclosure are detailed below.

The present disclosure also provides emissive polymeric matrix compositions of the materials described herein, which has the ability to polymerize to create solid or semi-solid matrices. The composition comprises at least one chromophore and at least one polymerisable monomer, such as hydroxyethylmethacrylate (HEMA), which in its polymerized form is referred to as poly hydroxyethylmethacrylate (pHEMA).

Suitable chromophores can be fluorescent compounds (or stains) (also known as “fluorochromes” or “fluorophores”). Other dye groups or dyes (biological and histological dyes, food colorings, carotenoids, and other dyes) can also be used. Advantageously, chromophores which are not well tolerated by the skin or other tissues can be included in the emissive polymeric matrices of the present disclosure, as in certain embodiments, the chromophores are encapsulated (e.g., into microspheres) within the emissive polymeric matrices and may not contact the tissues.

In certain embodiments, the emissive polymeric matrices of the present disclosure comprise a first chromophore which undergoes partial or complete photobleaching upon application of light. In some embodiments, the first chromophore absorbs at a wavelength in the range of the visible spectrum, such as at a wavelength of about 380 nm to about 800 nm, about 380 nm to about 700 nm, about 400 nm to about 800 nm, or about 380 nm to about 600 nm.

In other embodiments, the first chromophore absorbs at a wavelength of about 200 nm to about 800 nm, about 200 nm to about 700 nm, about 200 nm to about 600 nm or about 200 nm to about 500 nm. In one embodiment, the first chromophore absorbs at a wavelength of about 200 nm to about 600 nm. In some embodiments, the first chromophore absorbs light at a wavelength of about 200 nm to about 300 nm, about 250 nm to about 350 nm, about 300 nm to about 400 nm, about 350 nm to about 450 nm, about 400 nm to about 500 nm, about 450 nm to about 650 nm, about 600 nm to about 700 nm, about 650 nm to about 750 nm or about 700 nm to about 800 nm.

It will be appreciated to those skilled in the art that optical properties of a particular chromophore may vary depending on the chromophore's surrounding medium. Therefore, as used herein, a particular chromophore's absorption and/or emission wavelength (or spectrum) corresponds to the wavelengths (or spectrum) measured in an emissive polymeric matrices of the present disclosure.

The emissive polymeric matrices disclosed herein may include at least one additional chromophore. Combining chromophores may increase photo-absorption by the combined dye molecules and enhance absorption and photo-biomodulation selectivity. Thus, in certain embodiments, emissive polymeric matrices of the present disclosure include more than one chromophore. When such multi-chromophore materials are illuminated with light, energy transfer can occur between the chromophores. This process, known as resonance energy transfer, is a widely prevalent photophysical process through which an excited ‘donor’ chromophore (also referred to herein as first chromophore) transfers its excitation energy to an ‘acceptor’ chromophore (also referred to herein as second chromophore). The efficiency and directedness of resonance energy transfer depends on the spectral features of donor and acceptor chromophores. In particular, the flow of energy between chromophores is dependent on a spectral overlap reflecting the relative positioning and shapes of the absorption and emission spectra. More specifically, for energy transfer to occur, the emission spectrum of the donor chromophore must overlap with the absorption spectrum of the acceptor chromophore.

Energy transfer manifests itself through decrease or quenching of the donor emission and a reduction of excited state lifetime accompanied also by an increase in acceptor emission intensity. To enhance the energy transfer efficiency, the donor chromophore should have good abilities to absorb photons and emit photons. Furthermore, the more overlap there is between the donor chromophore's emission spectra and the acceptor chromophore's absorption spectra, the better a donor chromophore can transfer energy to the acceptor chromophore.

In certain embodiments, the emissive polymeric matrices of the present disclosure further comprise a second chromophore. In some embodiments, the first chromophore has an emission spectrum that overlaps at least about 80%, about 75%, about 70%, about 65%, about 60%, about 55%, about 50%, about 45%, about 40%, about 35%, about 30%, about 25%, about 20%, about 15% or about 10% with an absorption spectrum of the second chromophore. In one embodiment, the first chromophore has an emission spectrum that overlaps at least about 20% with an absorption spectrum of the second chromophore. In some embodiments, the first chromophore has an emission spectrum that overlaps at least between about 1% and about 10%, between about 5% and about 15%, between about 10% and about 20%, between about 15% and about 25%, between about 20% and about 30%, between about 25% and about 35%, between about 30% and about 40%, between about 35% and about 45%, between about 50% and about 60%, between about 55% and about 65% or between about 60% and about 70% with an absorption spectrum of the second chromophore.

% spectral overlap, as used herein, means the % overlap of a donor chromophore's emission wavelength range with an acceptor chromophore's absorption wavelength range, measured at spectral full width quarter maximum (FWQM). In some embodiments, the second chromophore absorbs at a wavelength in the range of the visible spectrum. In certain embodiments, the second chromophore has an absorption wavelength that is relatively longer than that of the first chromophore within the range of between about 50 and about 250, between about 25 and about 150 or between about 10 and about 100 nm.

The first chromophore can be present in an amount of about 0.01-0.9% per weight of the emissive polymeric matrices. When present, the second chromophore can be present in an amount of about 0.01-0.9% per weight of the emissive polymeric matrices.

The concentration of the chromophore to be used can be selected based on the desired intensity and duration of the biophotonic activity from the emissive polymeric matrix, and on the desired medical or cosmetic effect. For example, some dyes such as xanthene dyes reach a ‘saturation concentration’ after which further increases in concentration do not provide substantially higher emitted fluorescence. Further increasing the chromophore concentration above the saturation concentration can reduce the amount of activating light passing through the matrix. Therefore, if more fluorescence is required for a certain application than activating light, a high concentration of chromophore can be used. However, if a balance is required between the emitted fluorescence and the activating light, a concentration close to or lower than the saturation concentration can be chosen.

Suitable chromophores that may be used in the emissive polymeric matrices of the present disclosure include, but are not limited to the following:

Chlorophyll Dyes—

Exemplary chlorophyll dyes include but are not limited to chlorophyll a; chlorophyll b; chlorophyllin; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Xanthene Derivatives—

Exemplary xanthene dyes include but are not limited to eosin, eosin B (4′,5′-dibromo,2′,7′-dinitr-o-fluorescein, dianion); eosin Y; eosin Y (2′,4′,5′,7′-tetrabromo-fluoresc-ein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines that include, but are not limited to, 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

Methylene Blue Dyes—

Exemplary methylene blue derivatives include, but are not limited to, 1-methyl methylene blue; 1,9-dimethyl methylene blue; methylene blue; methylene blue (16 μM); methylene blue (14 μM); methylene violet; bromomethylene violet; 4-iodomethylene violet; 1,9-dimethyl-3-dimethyl-amino-7-diethyl-a-mino-phenothiazine; and 1,9-dimethyl-3-diethylamino-7-dibutyl-amino-phenot-hiazine.

Azo Dyes—

Exemplary azo (or diazo-) dyes include but are not limited to methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

In some aspects of the disclosure, the one or more chromophores comprised in the emissive polymeric matrices disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin Green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural green 3 (chlorophyllin), Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, Pyronin B, phycobilins, Phycocyanins, Phycoerythrins. Phycoerythrincyanin (PEC), Phthalocyanines, Pyronin G, Pyronin Y, Quinine, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Vitamin B, Water blue I, Water soluble eosin, Xylidine ponceau, or Yellowish eosin.

In certain embodiments, the emissive polymeric matrices of the present disclosure may include any of the chromophores listed above, or a combination thereof, so as to provide a synergistic biophotonic effect at the application site.

Without being bound to any particular theory, a synergistic effect of the chromophore combinations means that the biophotonic effect is greater than the sum of their individual effects. Advantageously, this may translate to increased reactivity of the emissive polymeric matrices, faster or improved treatment time. Also, the treatment conditions need not be altered to achieve the same or better treatment results, such as time of exposure to light, power of light source used, and wavelength of light used. In other words, use of synergistic combinations of chromophores may allow the same or better treatment without necessitating a longer time of exposure to a light source, a higher power light source or a light source with different wavelengths.

In some embodiments, the emissive polymeric matrix includes Eosin Y as a first chromophore and any one or more of Rose Bengal, Fluorescein, Erythrosine, Phloxine B, chlorophyllin as a second chromophore. In some implementations of these embodiments, the emissive polymeric matrix includes Eosin Y as a first chromophore and Fluorescein as a second chromophore.

It is believed that these combinations have a synergistic effect as they can transfer energy to one another when activated due in part to overlaps or close proximity of their absorption and emission spectra. This transferred energy may then be emitted as fluorescence and/or may lead to production of reactive oxygen species and/or may lead to production of radiant energy such as infrared. This absorbed and re-emitted light is thought to be transmitted throughout the matrix, and also to be transmitted into the site of treatment.

In further embodiments, the material includes the following synergistic combinations: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine. Other synergistic chromophore combinations are also possible.

By means of synergistic effects of the chromophore combinations in the emissive polymeric matrices, chromophores which cannot normally be activated by an activating light (such as a blue light from an LED), can be activated through energy transfer from chromophores which are activated by the activating light. In this way, the different properties of photoactivated chromophores can be harnessed and tailored according to the cosmetic or the medical therapy required.

For example, Rose Bengal can generate a high yield of singlet oxygen when activated in the presence of molecular oxygen, however it has a low quantum yield in terms of emitted fluorescent light. Rose Bengal has a peak absorption around 540 nm and so can be activated by green light. Eosin Y has a high quantum yield and can be activated by blue light. By combining Rose Bengal with Eosin Y, one obtains a composition which can emit therapeutic fluorescent light and generate singlet oxygen when activated by blue light. In this case, the blue light photoactivates Eosin Y which transfers some of its energy to Rose Bengal as well as emitting some energy as fluorescence.

In some embodiments, the chromophore or chromophores are selected such that their emitted fluorescent light, on photoactivation, is within one or more of the green, yellow, orange, red and infrared portions of the electromagnetic spectrum, for example having a peak wavelength within the range of about 490 nm to about 800 nm. In certain embodiments, the emitted fluorescent light has a power density of between 0.005 to about 15 mW/cm², about 0.5 to about 15 mW/cm².

### Polymerisable Monomers

In some implementations, the polymerisable monomer is a hydrophobic monomer. In some other implementations, the polymerisable monomer is a hydrophilic monomer. As used herein, a hydrophilic monomer refers to any monomer which, when polymerized, yields a hydrophilic polymer capable of forming a hydrogel when contacted with an aqueous medium such as water. In some instances, a hydrophilic monomer can contain a functional group in its backbone or as lateral chains. The term “functional group” as used herein refers to a chemical moiety which exhibits bond formation capability. Examples of functional group include, but are not limited to, hydroxyl (—OH), carboxyl (—COOH), amide (—CONH—), thiol (—SH), or sulfonic (—SO3H) groups. Examples of hydrophilic monomers include, but are not limited to, hydroxyl-containing monomers such as 2-hydroxyethyl methacrylate, 2-hydroxyethyl acrylate, 2-hydroxyethyl methacrylamide, 2-hydroxyethyl acrylamide, N-2-hydroxyethyl vinyl carbamate, 2-hydroxyethyl vinyl carbonate, 2-hydroxypropyl methacrylate, hydroxyhexyl methacrylate and hydroxyoctyl methacrylate; carboxyl-containing monomers such as acrylic acid, methacrylic acid, itaconic acid, fumaric acid, crotonic acid, maleic acid and salts thereof, esters containing free carboxyl groups of unsaturated polycarboxylic acids, such as monomethyl maleate ester, monoethyl maleate ester, monomethyl fumarate ester, monoethyl fumarate ester and salts thereof; amide-containing monomers such as (meth)acrylamide, crotonic amide, cinnamic amide, maleic diamide and fumaric diamide; thiol-containing monomers such as methanethiole, ethanethiol, 1-propanethiol, butanethiol, tert-butyl mercaptan, and pentanethiols; sulfonic acid-containing monomers such as p-styrenesulfonic acid, vinylsulfonic acid, p-a-methylstyrenesulfonic acid, isoprene sulfonide and salts thereof.

In certain aspects of the present disclosure the polymerisable monomer is 2-hydroxyethyl methacrylate (HEMA). In certain embodiments of the disclosure, the HEMA is present in the emissive polymeric matrix the amount of between about 1 wt % and about 80 wt %, or between about 5 wt % and about 80 wt %, or between about 10 wt % and about 80 wt %, or between about 15 wt % and about 80 wt %, or between about 5 wt % and about 50 wt %, or between about 10 wt % and about 50 wt %, or between about 15 wt % and about 50 wt %, or between about 15 wt % and about 50 wt %, or between about 5 wt % and about 30 wt %, or between about 10 wt % and about 30 wt %, or between about 15 wt % and about 30 wt %, or between about 15 wt % and about 25 wt % or about 20 wt % HEMA.

### Cross-Linkers

The cross-linking agent of the present disclosure is intended to form a cross-linked structure during the process of polymerization of the monomers defined herein. Typical examples of cross-linking agents include, but are not limited to, compounds having at least two polymerisable unsaturated double bonds in the molecular unit thereof, compounds having at least two groups capable of reacting with a functional group such as acid group, hydroxyl groups, amino group, in the molecule; compounds having at least one double bond and at least one group capable of reacting with the functional group of the monomer compounds having at least two points capable of reacting with the functional group of monomer within the molecular unit; and polymers capable of forming a cross-linked structure as by graft bondage during the process of polymerization of the monomer.

Some implementations of the emissive polymeric matrices of the present disclosure have a cross-linking agent including, but not limited to: ethylene glycol dimethacrylate (EGDMA); poly(ethylene glycol) diacrylate, or polyvalent(meth)acrylamide compounds such as N,N′-methylene bis(meth)acrylamide; or poly(meth)acrylate compounds such as poly(ethylene glycol) di(meth)acrylate, poly(propylene) glycol di(meth)acrylate, glycerol di(meth)acrylate, glycerol acrylate methacrylate, trimethylolpropane di (meth) acrylate, trimethylol propane acrylate methacrylate, pentaerythritol di(meth)acrylate, glycerol tri (meth) acrylate, trimethylolpropane tri (meth) acrylate, pentaerythritol tri(meth)acrylate, and pentaerythritol tetra-(meth)acrylate; or polyallyl compounds such as triallyl amine, poly(allyloxy) alkane, triallyl cyanurate, triallyl isocyanurate, and triallyl phosphate; or polyglycidyl compounds such as poly(ethylene glycol) diglycidyl ether, propylene glycol diglycidyl ether, glycerol diglycidyl ether, and glycerol triglycidyl ether; polyisocyanate compounds such as 2,4-toluylene diisocyanate and hexamethylene diisocyanate; polyoxazoline compounds; or reactive group-containing (meth)acryl amides or (meth)acrylates such as N-methylol (meth)acryl amide and glycidyl (meth)acrylate.

The density of cross-links adds to the absorption capacity and, at the same time, increases the content of soluble component. The amount of cross-linking agent employed in the current disclosure can be varied. In some implementations of the embodiments, the cross-linking agent is ethylene glycol dimethacrylate (EGDMA). In further implementations, the EGDMA is present in the emissive polymeric matrix composition in the amount of between about 0.1 wt % and about 10 wt %, or between about 1 wt % and about 5 wt % of the total composition.

### Initiators

Certain embodiments of the emissive polymeric matrices of the present disclosure may also comprise a polymerization initiator. As used herein, an “initiator” for a polymerization reaction refers to a compound that can initiate a polymerization reaction, typically by providing a free radical species. The free radical species can be generated directly by the initiator compound, or can be abstracted from a compound that facilitates initiation of polymerization. The free radicals generated or abstracted by the activated initiator compound can then propagate radical chain polymerization. Initiator molecules of the present disclosure may include, but are not limited to, free radical initiators. Examples of free radical initiators include, but are not limited to, organic peroxides, such as but not limited to, benzoyl peroxides. Other examples of organic peroxides that may be useful, include: tert-butyl hydroperoxide, tert-butyl peracetate, cumene hydroperoxide, 2,5-di(tert-butylperoxy)-2, 5-dimethyl-3-hexyne, 2,5-Di(tert-butylperoxy)-2,5-dimethyl-3-hexyne, dicumyl peroxide, 2,5-bis(tert-butylperoxy)-2,5-dimethylhexane, 2,4-pentanedione peroxide, 1,1-bis(tert-butylperoxy)-3,3,5-trimethylcyclohexane, 1,1-bis(tert-butylperoxy)cyclohexane, 1,1-bis(tert-amylperoxy)cyclohexane, 2-butanone peroxide, tert-butyl peroxide, lauroyl peroxide, tert-butyl peroxybenzoate, tert-butylperoxy 2-ethylhexyl carbonate, tert-butyl hydroperoxide.

In some embodiments of the emissive polymeric matrix compositions may comprise between about 0 wt % and about 1 wt %, between about 0.1 wt % and about 0.5 wt %, between about 0.2 wt % and about 1.0 wt %, between about 0.25 wt % and about 1.25 wt %, between about 0.1 wt % and about 2.0 wt %, between about 0.2 wt % and about 4.0 wt % benzoyl peroxide.

### Antimicrobials

Antimicrobials kill microbes or inhibit their growth or accumulation, and are optionally included in the emissive polymeric matrix of the present disclosure. Exemplary antimicrobials (or antimicrobial agent) are recited in U.S. Patent Application Publication Nos: 2004/0009227 and 2011/0081530. Suitable antimicrobials for use in the methods and compositions of the present disclosure include, but not limited to, hydrogen peroxide, urea hydrogen peroxide, benzoyl peroxide, phenolic and chlorinated phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

Hydrogen peroxide (H₂O₂) is a powerful oxidizing agent, and breaks down into water and oxygen and does not form any persistent, toxic residual compound. A suitable range of concentration over which hydrogen peroxide can be used in the emissive polymeric matrix is from about 0.1% to about 3%, about 0.1% to about 1.5%, about 0.1% to about 1%, about 1%, less than about 1%.

Urea hydrogen peroxide (also known as urea peroxide, carbamide peroxide or percarbamide) is soluble in water and contains approximately 35% hydrogen peroxide. A suitable range of concentration over which urea peroxide can be used in emissive polymeric matrix of the present disclosure is less than about 0.25%, or less than about 0.3%, from 0.001 to 0.25%, or from about 0.3% to about 5%. Urea peroxide breaks down to urea and hydrogen peroxide in a slow-release fashion that can be accelerated with heat or photochemical reactions.

Benzoyl peroxide consists of two benzoyl groups (benzoic acid with the H of the carboxylic acid removed) joined by a peroxide group. A suitable range of concentration over which benzoyl peroxide can be used in the emissive polymeric matrix is from about 2.5% to about 5%. According to certain embodiments, the emissive polymeric matrices of the present disclosure may optionally comprise one or more additional components, such as oxygen-rich compounds as a source of oxygen radicals. Peroxide compounds are oxidants that contain the peroxy group (R—O—O—R), which is a chainlike structure containing two oxygen atoms, each of which is bonded to the other and a radical or some element. When an emissive polymeric matrices of the present disclosure comprising an oxidant are illuminated with light, the chromophores are excited to a higher energy state. When the chromophores' electrons return to a lower energy state, they emit photons with a lower energy level, thus causing the emission of light of a longer wavelength (Stokes' shift). In the proper environment, some of this energy is transferred to oxygen or the reactive hydrogen peroxide and causes the formation of oxygen radicals, such as singlet oxygen. The singlet oxygen and other reactive oxygen species generated by the activation of the emissive polymeric matrices are thought to operate in a hormetic fashion. That is, a health beneficial effect that is brought about by the low exposure to a normally toxic stimuli (e.g. reactive oxygen), by stimulating and modulating stress response pathways in cells of the targeted tissues. Endogenous response to exogenous generated free radicals (reactive oxygen species) is modulated in increased defense capacity against the exogenous free radicals and induces acceleration of healing and regenerative processes. Furthermore, activation of the oxidant may also produce an antibacterial effect. The extreme sensitivity of bacteria to exposure to free radicals makes the emissive polymeric matrices of the present disclosure potentially a bactericidal composition.

### Optical Properties of the Emissive Polymeric Matrices

In certain embodiments, the emissive polymeric matrices of the present disclosure are substantially transparent or translucent. The % transmittance of the emissive polymeric matrices can be measured in the range of wavelengths from 250 nm to 800 nm using, for example, a Perkin-Elmer Lambda 9500 series UV-visible spectrophotometer. In some embodiments, transmittance within the visible range is measured and averaged. In some other embodiments, transmittance of the emissive polymeric matrices is measured with the chromophore omitted. As transmittance is dependent upon thickness, the thickness of each sample can be measured with calipers prior to loading in the spectrophotometer. Transmittance values can be normalized according to the formula:

$${F}_{T - corr} ( λ , {t}_{2} ) = {[ {}^{- {σ}_{t}} ( λ ) {t}_{1} ]}^{\frac{{t}_{2}}{{t}_{1}}} = {[ {F}_{T - corr} ( λ , {t}_{1} ) ]}^{\frac{{t}_{2}}{{t}_{1}}} ,$$

where t₁=actual specimen thickness, t₂=thickness to which transmittance measurements can be normalized. In the art, transmittance measurements are usually normalized to 1 cm.

In some embodiments, the emissive polymer matrix has a transmittance that is more than about 20%, 25%, 30%, 35%, 40%, 45%, 50%, 55%, 60%, 65%, 70%, or 75% within the visible range. In some embodiments, the transmittance exceeds 40%, 41%, 42%, 43%, 44%, or 45% within the visible range.

In some embodiments, the emissive polymeric matrices of the present disclosure comprise one or more additional component which assists in giving the matrix a particular consistency such as to impart to a solid emissive polymeric matrix more elasticity and/or more flexibility. Such a component may be, for example, a polyol (i.e., sugar alcohol). In some implementations, the one or more additional component may be glycerol. Other polyols that may be useful include, but are not limited to, arabitol, erythritol, isomalt, hydrogenated starch hydrolysates (HSH), lactitol, maltitol, mannitol, sorbitol, xylitol and sucrose.

### Methods of Use

The emissive polymeric matrices of the present disclosure may have cosmetic and/or medical benefits. They can be used to promote skin rejuvenation and skin conditioning, promote the treatment of a skin disorder such as acne, eczema, dermatitis or psoriasis, promote tissue repair, and modulate inflammation, modulate collagen synthesis, reduce or avoid scarring, for cosmesis, to promote wound healing including reducing the depth of periodontitis pockets. They can be used to treat acute inflammation. Acute inflammation can present itself as pain, heat, redness, swelling and loss of function, and includes inflammatory responses such as those seen in allergic reactions such as those to insect bites e.g.; mosquito, bees, wasps, poison ivy, or post-ablative treatment.

Accordingly, in certain embodiments, the present disclosure provides a method for treating acute inflammation.

In certain embodiments, the present disclosure provides a method for providing skin rejuvenation or for improving skin condition, treating a skin disorder, preventing or treating scarring, and/or accelerating wound healing and/or tissue repair, the method comprising: applying an emissive photonic matrix of the present disclosure to the area of the skin or tissue in need of treatment, and illuminating the emissive photonic matrix with light having a wavelength that overlaps with an absorption spectrum of the chromophore(s) present in the emissive polymeric matrix; and continued or repeated illumination of the emissive polymeric matrix with light having a wavelength that overlaps with an absorption spectrum of the chromophore(s) present in the emissive polymeric matrix.

In certain embodiments, the present disclosure provides a method for providing skin rejuvenation or for improving skin condition, treating a skin disorder, preventing or treating scarring, and/or accelerating wound healing and/or tissue repair, the method comprising: placing an emissive photonic matrix of the present disclosure in proximity of an area of the skin or tissue in need of treatment, and illuminating the emissive photonic matrix with light having a wavelength that overlaps with an absorption spectrum of the chromophore(s) present in the emissive polymeric matrix; and continued or repeated illumination of the emissive polymeric matrix with light having a wavelength that overlaps with an absorption spectrum of the chromophore(s) present in the emissive polymeric matrix.

In the methods of the present disclosure, any source of actinic light can be used. Any type of halogen, LED or plasma arc lamp or laser may be suitable. A characteristic of suitable sources of actinic light will be that they emit light in a wavelength (or wavelengths) appropriate for activating the one or more chromophores present in the composition. In one embodiment, an argon laser is used. In another embodiment, a potassium-titanyl phosphate (KTP) laser (e.g. a GreenLight™ laser) is used. In yet another embodiment, a LED lamp is the source of the actinic light. In yet another embodiment, the source of the actinic light is a source of light having a wavelength between about 200 nm to about 800 nm. In another embodiment, the source of the actinic light is a source of visible light having a wavelength between about 400 nm to about 600 nm. In another embodiment, the source of the actinic light is a source of visible light having a wavelength between about 400 nm to about 700 nm. In yet another embodiment, the source of the actinic light is blue light. In yet another embodiment, the source of the actinic light is red light. In yet another embodiment, the source of the actinic light is green light. Furthermore, the source of actinic light should have a suitable power density. Suitable power density for non-collimated light sources (LED, halogen or plasma lamps) are in the range from about 0.1 mW/cm² to about 200 mW/cm². Suitable power density for laser light sources are in the range from about 0.5 mW/cm² to about 0.8 mW/cm².

In some embodiments of the methods of the present disclosure, the light has an energy at the subject's skin surface of between about 0.1 mW/cm² and about 500 mW/cm², or 0.1-300 mW/cm², or 0.1-200 mW/cm², wherein the energy applied depends at least on the condition being treated, the wavelength of the light, the distance of the skin from the light source and the thickness of the biophotonic material. In certain embodiments, the light at the subject's skin is between about 1-40 mW/cm², or between about 20-60 mW/cm², or between about 40-80 mW/cm², or between about 60-100 mW/cm², or between about 80-120 mW/cm², or between about 100-140 mW/cm², or between about 30-180 mW/cm², or between about 120-160 mW/cm², or between about 140-180 mW/cm², or between about 160-200 mW/cm², or between about 110-240 mW/cm², or between about 110-150 mW/cm², or between about 190-240 mW/cm².

The activation of the chromophore(s) within the emissive polymeric matrix may take place almost immediately on illumination (femto- or pico seconds). A prolonged exposure period may be beneficial to exploit the synergistic effects of the absorbed, reflected and reemitted light of the emissive polymeric matrix of the present disclosure and its interaction with the tissue being treated. In one embodiment, the time of exposure of the tissue or skin or the emissive polymeric matrix to actinic light is a period between 0.01 minutes and 90 minutes. In another embodiment, the time of exposure of the tissue or skin or the emissive polymeric matrix to actinic light is a period between 1 minute and 5 minutes. In some other embodiments, the emissive polymeric matrix is illuminated for a period between 1 minute and 3 minutes. In certain embodiments, light is applied for a period of about 1-30 seconds, about 15-45 seconds, about 30-60 seconds, about 0.75-1.5 minutes, about 1-2 minutes, about 1.5-2.5 minutes, about 2-3 minutes, about 2.5-3.5 minutes, about 3-4 minutes, about 3.5-4.5 minutes, about 4-5 minutes, about 5-10 minutes, about 10-15 minutes, about 15-20 minutes, or about 20-30 minutes. The treatment time may range up to about 90 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area. For example, the delivered fluence may be about 4 to about 60 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm².

In certain embodiments, the emissive polymeric matrix may be re-illuminated at certain intervals. In yet another embodiment, the source of actinic light is in continuous motion over the treated area for the appropriate time of exposure.

In certain embodiments, the chromophore(s) in the emissive polymeric matrix can be photoexcited by ambient light including from the sun and overhead lighting. In certain embodiments, the chromophore(s) can be photoactivated by light in the visible range of the electromagnetic spectrum. The light can be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs, and indirect sunlight.

In the methods of the present disclosure, the emissive polymeric matrix may be removed from the skin following application of light. In other embodiments, the emissive polymeric matrix is left on the tissue for an extended period of time and re-activated with direct or ambient light at appropriate times to treat the condition.

In certain embodiments of the method of the present disclosure, the emissive polymeric matrix can be applied to the tissue or can be placed in proximity of the tissue, once, twice, three times, four times, five times or six times a week, daily, or at any other frequency. The total treatment time can be one week, two weeks, three weeks, four weeks, five weeks, six weeks, seven weeks, eight weeks, nine weeks, ten weeks, eleven weeks, twelve weeks, or any other length of time deemed appropriate.

In certain embodiments, the emissive polymeric matrix can be used to promote wound healing. In this case, the emissive polymeric matrix may be applied at wound site or may be placed in proximity of a wound site as deemed appropriate by the physician or other health care providers.

In certain embodiments, emissive polymeric matrix can be used following wound closure to optimize scar revision. In this case, the emissive polymeric matrix may be applied at the scar site to be treated or may be placed in proximity to the scar site to be treated at regular intervals such as once a week, or at an interval deemed appropriate by the physician or other health care providers.

In certain embodiments, the emissive polymeric matrix can be used following acne treatment to maintain the condition of the treated skin. In this case, the emissive polymeric matrix may be applied at regular intervals such as once a week, or at an interval deemed appropriate by the physician or other health care providers.

In certain embodiments, the emissive polymeric matrix can be used following ablative skin rejuvenation treatment to maintain the condition of the treated skin. In this case, the emissive polymeric matrix may be applied at regular intervals such as once a week, or at an interval deemed appropriate by the physician or other health care providers.

In the methods of the present disclosure, additional components may optionally be included in the emissive polymeric matrix or used in combination with the emissive polymeric matrix. Such additional components include, but are not limited to, healing factors, antimicrobials, oxygen-rich agents, wrinkle fillers such as botox, hyaluronic acid and polylactic acid, fungal, anti-bacterial, anti-viral agents and/or agents that promote collagen synthesis. These additional components may be applied to the skin in a topical fashion, prior to, at the same time of, and/or after topical application of emissive polymeric matrix of the present disclosure. Suitable healing factors comprise compounds that promote or enhance the healing or regenerative process of the tissues on the application site. During the photoactivation of an emissive polymeric matrix of the present disclosure, there may be an increase of the absorption of molecules of such additional components at the treatment site by the skin or the mucosa. In certain embodiments, an augmentation in the blood flow at the site of treatment can observed for a period of time. An increase in the lymphatic drainage and a possible change in the osmotic equilibrium due to the dynamic interaction of the free radical cascades can be enhanced or even fortified with the inclusion of healing factors. Healing factors may also modulate the biophotonic output from the biophotonic composition such as photobleaching time and profile, or modulate leaching of certain ingredients within the composition. Suitable healing factors include, but are not limited to glucosamines, allantoins, saffron, agents that promote collagen synthesis, anti-fungal, anti-bacterial, anti-viral agents and wound healing factors such as growth factors.

The emissive polymeric matrix of the present disclosure may be useful in promoting skin rejuvenation or improving skin condition and appearance. The dermis is the second layer of skin, containing the structural elements of the skin, the connective tissue. There are various types of connective tissue with different functions. Elastin fibers give the skin its elasticity, and collagen gives the skin its strength.

The junction between the dermis and the epidermis is an important structure. The dermal-epidermal junction interlocks forming finger-like epidermal ridges. The cells of the epidermis receive their nutrients from the blood vessels in the dermis. The epidermal ridges increase the surface area of the epidermis that is exposed to these blood vessels and the needed nutrients.

The aging of skin comes with significant physiological changes to the skin. The generation of new skin cells slows down, and the epidermal ridges of the dermal-epidermal junction flatten out. While the number of elastin fibers increases, their structure and coherence decreases. Also the amount of collagen and the thickness of the dermis decrease with the ageing of the skin.

Collagen is a major component of the skin's extracellular matrix, providing a structural framework. During the aging process, the decrease of collagen synthesis and insolubilization of collagen fibers contribute to a thinning of the dermis and loss of the skin's biomechanical properties.

The physiological changes to the skin result in noticeable aging symptoms often referred to as chronological-, intrinsic- and photo-aging. The skin becomes drier, roughness and scaling increase, the appearance becomes duller, and most obviously, fine lines and wrinkles appear. Other symptoms or signs of skin aging include, but are not limited to, thinning and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), an inability to sweat sufficiently to cool the skin, unwanted facial hair, freckles, age spots, spider veins, rough and leathery skin, fine wrinkles that disappear when stretched, loose skin and/or a blotchy complexion.

The dermal-epidermal junction is a basement membrane that separates the keratinocytes in the epidermis from the extracellular matrix, which lies below in the dermis. This membrane consists of two layers: the basal lamina in contact with the keratinocytes, and the underlying reticular lamina in contact with the extracellular matrix. The basal lamina is rich in collagen type IV and laminin, molecules that play a role in providing a structural network and bioadhesive properties for cell attachment.

Laminin is a glycoprotein that only exists in basement membranes. It is composed of three polypeptide chains (alpha, beta and gamma) arranged in the shape of an asymmetric cross and held together by disulfide bonds. The three chains exist as different subtypes which result in twelve different isoforms for laminin, including Laminin-1 and Laminin-5.

The dermis is anchored to hemidesmosomes, specific junction points located on the keratinocytes, which consist of α-integrins and other proteins, at the basal membrane keratinocytes by type VII collagen fibrils. Laminins, and particularly Laminin-5, constitute the real anchor point between hemidesmosomal transmembrane proteins in basal keratinocytes and type VII collagen.

Laminin-5 synthesis and type VII collagen expression have been proven to decrease in aged skin. This causes a loss of contact between dermis and epidermis, and results in the skin losing elasticity and becoming saggy.

Recently another type of wrinkles, generally referred to as expression wrinkles, received general recognition. Expression wrinkles result from a loss of resilience, particularly in the dermis, because of which the skin is no longer able to resume its original state when facial muscles which produce facial expressions.

The emissive polymeric matrices of the present disclosure and methods of the present disclosure may be used to promote skin rejuvenation. In certain embodiments, the emissive polymeric matrices and methods of the present disclosure may be used to promote skin luminosity, reduction of pore size, reducing blotchiness, making even skin tone, reducing dryness, and tightening of the skin, thereby promoting skin rejuvenation. In certain embodiments, the emissive polymeric matrices and methods of the present disclosure promote collagen synthesis. In certain other embodiments, the emissive polymeric matrices and methods of the present disclosure may reduce, diminish, retard or even reverse one or more signs of skin aging including, but not limited to, appearance of fine lines or wrinkles, thin and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), an inability to sweat sufficiently to cool the skin, unwanted facial hair, freckles, age spots, spider veins, rough and leathery skin, fine wrinkles that disappear when stretched, loose skin, or a blotchy complexion. In certain embodiments, the emissive polymeric matrices and methods of the present disclosure may induce a reduction in pore size, enhance sculpturing of skin subsections, and/or enhance skin translucence.

In certain embodiments, the emissive polymeric matrices may be used in conjunction with collagen promoting agents. Agents that promote collagen synthesis (i.e., pro-collagen synthesis agents) include amino acids, peptides, proteins, lipids, small chemical molecules, natural products and extracts from natural products.

For instance, it was discovered that intake of vitamin C, iron, and collagen can effectively increase the amount of collagen in skin or bone. See, e.g., U.S. Patent Application Publication 2009/0069217. Examples of the vitamin C include an ascorbic acid derivative such as L-ascorbic acid or sodium L-ascorbate, an ascorbic acid preparation obtained by coating ascorbic acid with an emulsifier or the like, and a mixture containing two or more of those vitamin Cs at an arbitrary rate. In addition, natural products containing vitamin C such as acerola or lemon may also be used. Examples of the iron preparation include: an inorganic iron such as ferrous sulfate, sodium ferrous citrate, or ferric pyrophosphate; an organic iron such as heme iron, ferritin iron, or lactoferrin iron; and a mixture containing two or more of those irons at an arbitrary rate. In addition, natural products containing iron such as spinach or liver may also be used. Moreover, examples of the collagen include: an extract obtained by treating bone, skin, or the like of a mammal such as bovine or swine with an acid or alkaline; a peptide obtained by hydrolyzing the extract with a protease such as pepsin, trypsin, or chymotrypsin; and a mixture containing two or more of those collagens at an arbitrary rate. Collagens extracted from plant sources may also be used.

Additional pro-collagen synthesis agents are described, for example, in U.S. Pat. Nos. 7,598,291; 7,722,904; 6,203,805; 5,529,769; and U.S. Patent Application Publications Nos: 2006/0247313; 2008/0108681; 2011/0130459; 2009/0325885; and 2011/0086060.

The emissive polymeric matrices and methods of the present disclosure may be used to treat skin disorders that include, but are not limited to, erythema, telangiectasia, actinic telangiectasia, basal cell carcinoma, contact dermatitis, dermatofibrosarcoma protuberans, genital warts, hidradenitis suppurativa, melanoma, merkel cell carcinoma, nummular dermatitis, molloscum contagiosum, psoriasis, psoriatic arthritis, rosacea, scabies, scalp psoriasis, sebaceous carcinoma, squamous cell carcinoma, seborrheic dermatitis, seborrheic keratosis, shingles, tinea versicolor, warts, skin cancer, pemphigus, sunburn, dermatitis, eczema, rashes, impetigo, lichen simplex chronicus, rhinophyma, perioral dermatitis, pseudofolliculitis barbae, drug eruptions, erythema multiforme, erythema nodosum, granuloma annulare, actinic keratosis, purpura, alopecia areata, aphthous stomatitis, dry skin, chapping, xerosis, ichthyosis vulgaris, fungal infections, herpes simplex, intertrigo, keloids, keratoses, milia, moluscum contagiosum, pityriasis rosea, pruritus, urticaria, and vascular tumors and malformations. Dermatitis includes contact dermatitis, atopic dermatitis, seborrheic dermatitis, nummular dermatitis, generalized exfoliative dermatitis, and statis dermatitis. Skin cancers include melanoma, basal cell carcinoma, and squamous cell carcinoma.

The emissive polymeric matrices and methods of the present disclosure may be used to treat acne. As used herein, “acne” means a disorder of the skin caused by inflammation of skin glands or hair follicles. The emissive polymeric matrices and methods of the disclosure can be used to treat acne at early pre-emergent stages or later stages where lesions from acne are visible. Mild, moderate and severe acne can be treated with embodiments of emissive polymeric matrices and methods. Early pre-emergent stages of acne usually begin with an excessive secretion of sebum or dermal oil from the sebaceous glands located in the pilosebaceous apparatus. Sebum reaches the skin surface through the duct of the hair follicle. The presence of excessive amounts of sebum in the duct and on the skin tends to obstruct or stagnate the normal flow of sebum from the follicular duct, thus producing a thickening and solidification of the sebum to create a solid plug known as a comedone. In the normal sequence of developing acne, hyperkeratinazation of the follicular opening is stimulated, thus completing blocking of the duct. The usual results are papules, pustules, or cysts, often contaminated with bacteria, which cause secondary infections. Acne is characterized particularly by the presence of comedones, inflammatory papules, or cysts. The appearance of acne may range from slight skin irritation to pitting and even the development of disfiguring scars. Accordingly, the emissive polymeric matrices and methods of the present disclosure can be used to treat one or more of skin irritation, pitting, development of scars, comedones, inflammatory papules, cysts, hyperkeratinazation, and thickening and hardening of sebum associated with acne.

Some skin disorders present various symptoms including redness, flushing, burning, scaling, pimples, papules, pustules, comedones, macules, nodules, vesicles, blisters, telangiectasia, spider veins, sores, surface irritations or pain, itching, inflammation, red, purple, or blue patches or discolorations, moles, and/or tumors.

The emissive polymeric matrices and methods of the present disclosure may be used to treat various types of acne. Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, and nodulocystic acne.

In certain embodiments, the emissive polymeric matrices of the present disclosure may be used in conjunction with systemic or topical antibiotic treatment. For example, antibiotics used to treat acne include tetracycline, erythromycin, minocycline, doxycycline, which may also be used with the emissive polymeric matrices and methods of the present disclosure. The use of the emissive polymeric matrices can reduce the time needed for the antibiotic treatment or reduce the dosage.

The emissive polymeric matrices and methods of the present disclosure may be used to treat wounds, promote wound healing, promote tissue repair and/or prevent or reduce cosmesis including improvement of motor function (e.g. movement of joints). Wounds that may be treated by the biophotonic hydrogels and methods of the present disclosure include, for example, injuries to the skin and subcutaneous tissue initiated in different ways (e.g., pressure ulcers from extended bed rest, wounds induced by trauma or surgery, burns, ulcers linked to diabetes or venous insufficiency, wounds induced by conditions such as periodontitis) and with varying characteristics. In certain embodiments, the present disclosure provides emissive polymeric matrices and methods for treating and/or promoting the healing of, for example, burns, incisions, excisions, lesions, lacerations, abrasions, puncture or penetrating wounds, surgical wounds, contusions, hematomas, crushing injuries, amputations, sores and ulcers.

The emissive polymeric matrices and methods of the present disclosure may be used to treat and/or promote the healing of chronic cutaneous ulcers or wounds, which are wounds that have failed to proceed through an orderly and timely series of events to produce a durable structural, functional, and cosmetic closure. The vast majority of chronic wounds can be classified into three categories based on their etiology: pressure ulcers, neuropathic (diabetic foot) ulcers and vascular (venous or arterial) ulcers.

For example, the present disclosure provides the emissive polymeric matrices and methods for treating and/or promoting healing of a diabetic ulcer. Diabetic patients are prone to foot and other ulcerations due to both neurologic and vascular complications. Peripheral neuropathy can cause altered or complete loss of sensation in the foot and/or leg. Diabetic patients with advanced neuropathy lose all ability for sharp-dull discrimination. Any cuts or trauma to the foot may go completely unnoticed for days or weeks in a patient with neuropathy. A patient with advanced neuropathy loses the ability to sense a sustained pressure insult, as a result, tissue ischemia and necrosis may occur leading to for example, plantar ulcerations. Microvascular disease is one of the significant complications for diabetics which may also lead to ulcerations. In certain embodiments, the emissive polymeric matrices and methods of treating a chronic wound are provided herein, where the chronic wound is characterized by diabetic foot ulcers and/or ulcerations due to neurologic and/or vascular complications of diabetes.

In other examples, the present disclosure provides emissive polymeric matrices and methods for treating and/or promoting healing of a pressure ulcer. Pressure ulcers include bed sores, decubitus ulcers and ischial tuberosity ulcers and can cause considerable pain and discomfort to a patient. A pressure ulcer can occur as a result of a prolonged pressure applied to the skin. Thus, pressure can be exerted on the skin of a patient due to the weight or mass of an individual. A pressure ulcer can develop when blood supply to an area of the skin is obstructed or cut off for more than two or three hours. The affected skin area can turn red, become painful and necrotic. If untreated, the skin can break open and become infected. A pressure ulcer is therefore a skin ulcer that occurs in an area of the skin that is under pressure from e.g. lying in bed, sifting in a wheelchair, and/or wearing a cast for a prolonged period of time. Pressure ulcers can occur when a person is bedridden, unconscious, unable to sense pain, or immobile. Pressure ulcers often occur in honey prominences of the body such as the buttocks area (on the sacrum or iliac crest), or on the heels of foot.

Additional types of wounds that can be treated by the emissive polymeric matrices and methods of the present disclosure include those disclosed by U.S. Pat. Appl. Publ. No. 2009/0220450, which is incorporated herein by reference.

There are three distinct phases in the wound healing process. First, in the inflammatory phase, which typically occurs from the moment a wound occurs until the first two to five days, platelets aggregate to deposit granules, promoting the deposit of fibrin and stimulating the release of growth factors. Leukocytes migrate to the wound site and begin to digest and transport debris away from the wound. During this inflammatory phase, monocytes are also converted to macrophages, which release growth factors for stimulating angiogenesis and the production of fibroblasts.

Second, in the proliferative phase, which typically occurs from two days to three weeks from wound occurrence, granulation tissue forms, and epithelialization and contraction begin. Fibroblasts, which are key cell types in this phase, proliferate and synthesize collagen to fill the wound and provide a strong matrix on which epithelial cells grow. As fibroblasts produce collagen, vascularization extends from nearby vessels, resulting in granulation tissue. Granulation tissue typically grows from the base of the wound. Epithelialization involves the migration of epithelial cells from the wound surfaces to seal the wound. Epithelial cells are driven by the need to contact cells of like type and are guided by a network of fibrin strands that function as a grid over which these cells migrate. Contractile cells called myofibroblasts appear in wounds, and aid in wound closure. These cells exhibit collagen synthesis and contractility, and are common in granulating wounds.

Third, in the remodeling phase, the final phase of wound healing which can take place from three weeks up to several years from wound occurrence, collagen in the scar undergoes repeated degradation and re-synthesis. During this phase, the tensile strength of the newly formed skin increases.

However, as the rate of wound healing increases, there is often an associated increase in scar formation. Scarring is a consequence of the healing process in most adult animal and human tissues. Scar tissue is not identical to the tissue which it replaces, as it is usually of inferior functional quality. The types of scars include, but are not limited to, atrophic, hypertrophic and keloidal scars, as well as scar contractures. Atrophic scars are flat and depressed below the surrounding skin as a valley or hole. Hypertrophic scars are elevated scars that remain within the boundaries of the original lesion, and often contain excessive collagen arranged in an abnormal pattern. Keloidal scars are elevated scars that spread beyond the margins of the original wound and invade the surrounding normal skin in a way that is site specific, and often contain whorls of collagen arranged in an abnormal fashion.

In contrast, normal skin consists of collagen fibers arranged in a basket-weave pattern, which contributes to both the strength and elasticity of the dermis. Thus, to achieve a smoother wound healing process, an approach is needed that not only stimulates collagen production, but also does so in a way that reduces scar formation.

The emissive polymeric matrices and methods of the present disclosure may promote the wound healing by promoting the formation of substantially uniform epithelialization; promoting collagen synthesis; promoting controlled contraction; and/or by reducing the formation of scar tissue. In certain embodiments, the emissive polymeric matrices and methods of the present disclosure may promote wound healing by promoting the formation of substantially uniform epithelialization. In some embodiments, the emissive polymeric matrices and methods of the present disclosure promote collagen synthesis. In some other embodiments, the emissive polymeric matrices and methods of the present disclosure promote controlled contraction. In certain embodiments, the emissive polymeric matrices and methods of the present disclosure promote wound healing, for example, by reducing the formation of scar tissue.

In the methods of the present disclosure, the emissive polymeric matrices of the present disclosure may also be used in combination with negative pressure assisted wound closure devices and systems.

In certain embodiments, the emissive polymeric matrices may be kept in place for up to one, two or 3 weeks, and illuminated with light which may include ambient light at various intervals. In this case, the composition may be covered up in between exposure to light with an opaque material or left exposed to light.

Rare diseases in dermatology for which the emissive polymeric matrices of the present disclosure may be used to treat or alleviate one or more symptoms thereof may include, but are not limited to, CHILD syndrome and in particular the ichthyosiform erythroderma aspect of CHILD syndrome; dermatomyositis; hidradenitis suppurativa; acquired ichthyosis as well as hereditary ichthyosis; lichen myxedematosus and scleromyxedema; pemphigus; and porphyria disorders.

Rare diseases involving bone and/or connective tissue maladies for which the emissive polymeric matrices of the present disclosure may be used to treat or alleviate one or more symptoms thereof may include, but are not limited to, Ehlers-Danlos syndrome and other rare diseases manifested by a collagen production and/or deposition abnormality; cutis hyperelastica; eosinophilic fasciitis; osteogenesis imperfecta; scelroderma; and Winchester syndrome.

Other rare dermatological, bone, connective tissue and even oral diseases for which the present invention may be used to treat or alleviate one or more symptoms thereof may also be found by reference to Touitou et al. (2013) “The expanding spectrum of rare monogenic autoinflammatory diseases” Orphan Journal of Rare Diseases, volume 8, pages 162-174.

The emissive polymeric matrices of the present disclosure may be used to treat various oral diseases. Such oral diseases include but are not limited to: gingivitis which is a disorder that is defined by the inflammation of the gums, and is characterized as a periodontal disease characterized by the destruction of the gums, tissue, tooth sockets, and ligaments which create the structure that holds the teeth in place. Gingivitis is one of the first stages of serious periodontal disease. The symptoms of gingivitis include swollen gums, mouth sores, a bright red or purple appearance to the gums, shiny gums, gums that are painless except when touched, and bleeding gums. Often the first signs of gingivitis have no symptoms except for visual symptoms and are likely only to be diagnosed by a dental professional.

Periodontal disease is also known as trench mouth. Periodontal disease leads to severe gingivitis and can cause gums to bleed, ooze pus, is highly painful, and often leads to premature tooth loss. Symptoms of periodontal disease include painful gums, bad breath (halitosis), a foul taste to the mouth, fever, gums that bleed with only mild amounts of pressure, crater sized canker sores between the teeth and gums, swollen lymph nodes around the head, neck, or jaw, a gray film on the gums, red gums, swollen gums, and pain when eating and swallowing. Periodontitis or Pyorrhea alveolaris is the inflammation of the periodontium which comprises tissues supporting the teeth in the oral cavity. Parts included in the periodontium are the gingiva (gum tissue), the alveolar bone which are sockets where teeth are attached, the cementum or outer layer of teeth roots and the periodontal ligaments or PDL composed of connective tissue fibers linking the gingival and cementum to the alveolar bone. The condition is described as the progressive loss of bone around teeth leading to loose teeth or loss of teeth if left unattended. There are different causes for the disease in which bacteria is the most common. Periodontitis is considered as an advanced phase of gum disease since it already involves bone loss in the area. It is the effect of mild gingivitis being left untreated. Due to the presence of bacterial infection, the body can also respond negatively to it leading to further complications. The condition is one of the leading causes of tooth loss among adults, affecting around 50% of everyone over the age of 30.

Signs and symptoms arise due to the unstable anchoring of teeth as well as the presence of microorganisms. Gums occasionally or frequently bleed or turn red while brushing teeth, using dental floss, biting into food, chewing or touching with fingers. Gums swell or develop pus occasionally as well. The affected individual likely has halitosis or bad breath and have a lingering metallic or tinny taste inside the mouth. Teeth seem longer and sharper due to gingival recession which partly may also be caused by hard brushing. If enzymes called collagenases have begun destroying collagen, the person will have deep pockets between the teeth and gums. During the early stages of periodontal disease, only a few signs and symptoms may be noticeable. Aggressive periodontitis may affect younger individuals and can occur in episodes. Some episodes may present very mild symptoms while others may be very severe. The signs and symptoms especially in the case of chronic periodontitis are usually progressive in nature.

Oral thrush is the condition where the fungus Candida albicans grows rapidly and uncontrollably in the mouth. The bacterium known as flora keeps the growth of Candida albicans under control in a healthy body. Oral thrush presents with creamy white paste that covers the tongue, and can spread rapidly to the roof of the mouth, gums, back of the throat, tonsils, and the inside of the cheeks. Babies, toddlers, older adults, and patients whose immune systems have been somehow compromised are most likely to come down with oral thrush.

Symptoms of oral thrush begin with a white pasty covering over the tongue and inside of the cheeks. As the oral thrush continues to develop, it can cause a mild amount of bleeding if the tongue is scraped or when the patient brushes their teeth. These symptoms may develop very quickly, but thrush can last for months. If the lesions of oral thrush spread down the esophagus, the patient may develop addition symptoms such as difficulty swallowing, the sensation of food being caught in the throat or the middle of the chest, and a fever should the infection continue to spread past the esophagus.

Lichen planus is most often defined as an oral disease that affects the lining of the mouth with inflammation. Lichen planus is most often recognized as a rash that irritates the tissue of the oral cavity. Most patients come down with their first case between the age of 45 and 60, although a slowly increasing number of reports dealing with younger patients have trickled in. While lichen planus is most often associated with the interior of the cheeks, many cases will find the entire mouth is affected, including the gums, the tongue, the lips, and in rare cases, the throat or esophagus. Lichen planus also occurs on the skin, as a skin disease, and often must be referred to specifically as skin lichen planus to differentiate between the oral type.

Lichen planus is a self-contained disease that can last for weeks, months, and in some cases, years. It is not contagious. It is often mistaken for genital diseases, as the genitalia are often the most noticeably affected during the early development stage. Because the symptoms and outbreaks occur rapidly and then disappear, often for weeks, treatment is difficult. While some patients find great relief in cool compresses or tub soaks and cool baths, most patients require medical treatment in order to relieve their symptoms.

Stomatitis basically means inflammation of the mouth, but more specifically, stomatitis is the inflammation of the mucous lining of the mouth which may include the gums, tongue, cheeks, lips and the floor or roof of the mouth. There are different types of stomatitis and classification is based on how the disease was acquired by a person. The two types of stomatitis are contact stomatitis and aphthous stomatitis. Contact stomatitis is an inflammation of the oral mucosa caused by coming in contact with allergens or irritants. It is classified by its pattern of distribution, etiologic factors, and clinical features. There some cases of contact stomatitis that are left undetected because of the lack of clinical signs. Anybody can have contact stomatitis regardless of race, age and sex. Although it has been observed that it is more common in the elders.

Aphthous stomatitis, also known as canker sore or aphthous ulcers, has an unknown etiology. Just like contact stomatitis, canker sore affects the oral mucosa. An aphthous ulcer is a type of oral ulcer, which presents as a painful open sore inside the mouth or upper throat (including the uvula) caused by a break in the mucous membrane. The condition is also known as Sutton's Disease, especially in the case of major, multiple, or recurring ulcers. The ulcers can be described as shallow, discrete, and painful and are usually visible on the mucous membranes that are unattached. This type of stomatitis, just like contact stomatitis, is self-limited and do not usually cause complications. The normal size of ulcers may last for 1 to 2 weeks but larger ulcers may last for months.

Herpes simplex is a viral disease caused by herpes simplex viruses; both herpes simplex virus 1 (HSV-1) and herpes simplex virus 2 (HSV-2) cause herpes simplex. Infection with the herpes virus is categorized into one of several distinct disorders based on the site of infection. Oral Herpes, the visible symptoms of which are colloquially called cold sores, and infects the face and mouth. Oral herpes is the most common form of herpes simplex virus infection.

The present invention may be used to treat other types of oral inflammation, including but not limited to oral mucositis, oral ulcers caused by viral, bacterial, fungal or protozoan infections, or caused by disorders of the immune system (immunodeficiency, autoimmunity, or allergy). Also included is Oral Submucous Fibrosis, a chronic debilitating disease of the oral cavity characterized by inflammation and progressive fibrosis of the submucosal tissues. Also included is Glossitis, an inflammation or infection of the tongue. It causes the tongue to swell and change color.

The emissive polymeric matrices of the present disclosure may be useful for bone reconstruction and/or regeneration. Without being bound by theory, the emissive polymeric matrices of the disclosure may help promote the growth, recruitment and survival of bone tissue at a particular site. In use, the emissive polymeric matrices may be implanted at a site at which bone growth is desired, e.g. to treat a disease, defect or location of trauma, and/or to promote artificial arthrodesis. Bone repair sites that can be treated with the composition of the disclosure include, but are not limited to, those resulting from injury, defects brought about during the course of surgery, infection, malignancy or developmental malformation. The emissive polymeric matrices can be used in a wide variety of orthopedic, periodontal, neurosurgical and oral and maxillofacial surgical procedures including, but not limited to: the repair of simple and compound fractures and non-unions; external and internal fixations; joint reconstructions such as arthrodesis; general arthroplasty; cup arthroplasty of the hip; femoral and humeral head replacement; femoral head surface replacement and total joint replacement; repairs of the vertebral column including spinal fusion and internal fixation; tumor surgery, e.g., deficit filing; discectomy; laminectomy; excision of spinal cord tumors; anterior cervical and thoracic operations; repairs of spinal injuries; scoliosis, lordosis and kyphosis treatments; intermaxillary fixation of fractures; mentoplasty; temporomandibular joint replacement; alveolar ridge augmentation and reconstruction; inlay osteoimplants; implant placement and revision; sinus lifts; cosmetic enhancement; etc. For any of these potential applications, the emissive polymeric matrices of the disclosure may be applied directly to a site where bone reconstruction is needed. Accessing this site may, in some cases, require surgical intervention to expose the site. However, in some cases, the site is already exposed or can be accessed without the need for surgical intervention.

Any bone disease or disorder may be treated using the emissive polymeric matrices of the present disclosure including genetic diseases, congenital abnormalities, fractures, iatrogenic defects, bone cancer, bone metastases, inflammatory diseases (e.g. rheumatoid arthritis), autoimmune diseases, metabolic diseases, and degenerative bone disease (e.g., osteoarthritis). In certain embodiments, the emissive polymeric matrices are formulated for the repair of a simple fracture, compound fracture, or non-union; as an external fixation device or internal fixation device; for joint reconstruction, arthrodesis, arthroplasty, or cup arthroplasty of the hip; for femoral or humeral head replacement; for femoral head surface replacement or total joint replacement; for repair of the vertebral column, spinal fusion or internal vertebral fixation; for tumor surgery; for deficit filling; for discectomy; for laminectomy; for excision of spinal tumors; for an anterior cervical or thoracic operation; for the repairs of a spinal injury; for scoliosis, for lordosis or kyphosis treatment; for intermaxillary fixation of a fracture; for mentoplasty; for temporomandibular joint replacement; for alveolar ridge augmentation and reconstruction; as an inlay osteoimplant; for implant placement and revision; for sinus lift; for a cosmetic procedure; for revision surgery; for revision surgery of a total joint arthroplasty; and for the repair or replacement of the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal bones, or metatarsal bones.

### Kit

The present disclosure also provides kits for preparing and/or providing any of the components required for forming emissive polymeric matrices of the present disclosure. In some embodiments, the kit includes containers comprising the components or compositions that can be used to make the emissive polymeric matrices of the present disclosure. In some embodiments, the kit includes emissive polymeric matrices material of the present disclosure. The different components making up the emissive polymeric matrices of the present disclosure may be provided in separate containers. For example, the polymerisable monomer may be provided in a container separate from the chromophore. Examples of such containers are dual chamber syringes, dual chamber containers with removable partitions, sachets with pouches, and multiple-compartment blister packs. Another example is one of the components being provided in a syringe which can be injected into a container of another component.

In other embodiments, the kit comprises a systemic drug for augmenting the treatment of the emissive polymeric matrices of the present disclosure. For example, the kit may include a systemic or topical antibiotic, hormone treatment (e.g. for acne treatment or wound healing), or a negative pressure device.

In other embodiments, the kit comprises a means for applying the components of the emissive polymeric matrices.

In certain aspects, there is provided a container comprising a chamber for holding emissive polymeric matrices, and an outlet in communication with the chamber for discharging the biophotonic material from the container, wherein the emissive polymeric matrices comprises at least one chromophore.

In other embodiments, the kit comprises the emissive polymeric matrices as well as instructions on how to use the emissive polymeric matrices.

In certain embodiments of the kit, the kit may further comprise a light source such as a portable light with a wavelength appropriate to activate the chromophore of the emissive polymeric matrices. The portable light may be battery operated or re-chargeable.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein. All references cited herein are incorporated by reference in their entirety and made part of this application.

Practice of the disclosure will be still more fully understood from the following examples, which are presented herein for illustration only and should not be construed as limiting the disclosure in any way.

### EXAMPLES

### Example 1

### Preparation of a Solid Disk of Polymerized HEMA Incorporating Eosin Y and Fluorescein

A solid disk of polymerized HEMA incorporating Eosin Y and Fluorescein was prepared by mixing together the following materials in the order given below:

1) Pure water-Hyclone (30 ml) #SH30529.02 from Thermo Scientific;
2) Benzoyl peroxide (100 mg) (BPO) #517909 from Sigma Aldrich Co. USA, which was used as initiator;
3) Eosin Y (50 mg) D&C Red22 #2012-27447, from Spectra Colors Corporation;
4) Fluorescein (50 mg) D&C Yellow 8 #2012-27110, from Spectra Colors Corporation;
5) 2-Hydroxyethyl methacrylate (70 ml) (HEMA) #477028, from Sigma Aldrich Co. USA; and
6) Ethylene glycol dimethacrylate (500 μl) (EGDMA) #335681, from Sigma Aldrich Co. USA, which was used as cross-linker.

Eosin Y and Fluorescein were added to liquid monomer pre-polymerization.

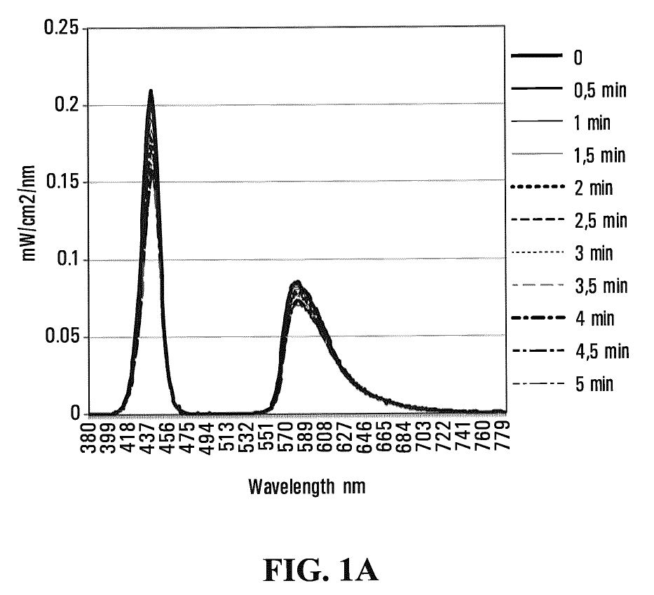

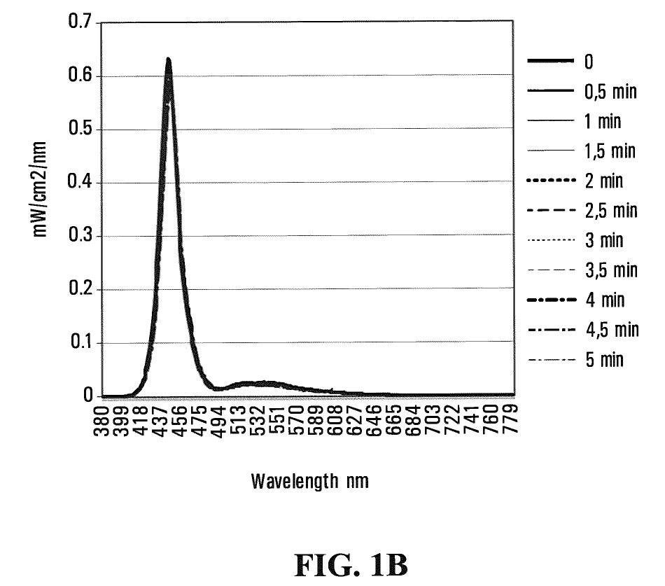

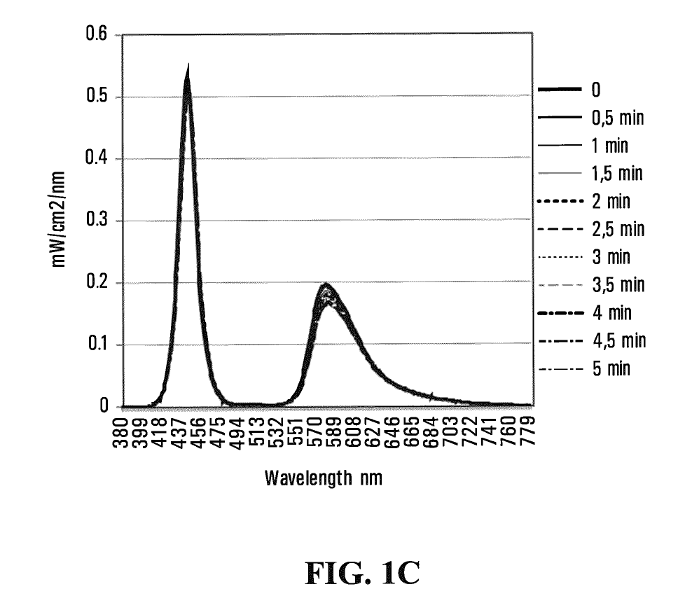

Polymerization was carried out in a nitrogen atmosphere for 5 hours at 70° C. The mixture was placed in a mould (glass Erlenmeyer or plastic container) under pressure at 76° C. for a period of between 6 and 8 hours. FIG. 1A shows the emission spectra of a 4 mm thick pHEMA transparent disk during 5 minutes of illumination. FIG. 1B shows the light emission spectra of a 2 mm thick pHEMA transparent disc during 5 minutes of illumination. FIG. 1C shows the light emission spectra of a 2.3 mm thick pHEMA disk during 5 minutes of illumination after a storage period of 5 minutes. Tables 1A, 1B and 1C below show the emission data corresponding to FIGS. 1A, 1B and 1C respectively.

TABLE 1A

| | | mW/cm2 at 5 cm | | | | | | |
|---|---|---|---|---|---|---|---|---|
| phema 4 mm disk translucid (E50 + F50) | | 0 | 0.5 min | 1 min | 1.5 min | 2 min | 2.5 min | 3 min |
| lamp | 400-518 | 4.41 | 4.27 | 4.14 | 4.00 | 3.86 | 3.74 | 3.64 |
| Fluoresc. | 519-760 | 4.80 | 4.73 | 4.69 | 4.63 | 4.55 | 4.47 | 4.41 |
| total | 400-760 | 9.209571 | 9.003722 | 8.826337 | 8.626539 | 8.412343 | 8.209865 | 8.040929 |
| % fluorescence | | 52.1% | 52.6% | 53.1% | 53.6% | 54.1% | 54.5% | 54.8% |
| purple | (400)-450 | 3.9324 | 3.7854 | 3.6615 | 3.5296 | 3.4002 | 3.2810 | 3.1809 |
| Blue | 450-500 | 0.4743 | 0.4833 | 0.4784 | 0.4708 | 0.4623 | 0.4561 | 0.4537 |
| Green | 500-570 | 0.4481 | 0.4336 | 0.4127 | 0.3936 | 0.3751 | 0.3586 | 0.3456 |
| Yellow | 570-591 | 1.6899 | 1.6604 | 1.6298 | 1.5972 | 1.5625 | 1.5270 | 1.4941 |
| Orange | 591-610 | 1.2819 | 1.2543 | 1.2416 | 1.2372 | 1.2069 | 1.1863 | 1.1682 |
| Red | 610-760 | 1.4332 | 1.4353 | 1.4505 | 1.4557 | 1.4524 | 1.4474 | 1.4438 |
| total | (400-700) | 9.26 | 9.05 | 8.87 | 8.67 | 8.46 | 8.26 | 8.09 |

| | | mW/cm2 at 5 cm | | | | | |
|---|---|---|---|---|---|---|---|
| phema 4 mm disk translucid (E50 + F50) | | 3.5 min | 4 min | 4.5 min | 5 min | J/cm2 | |
| lamp | 400-518 | 3.54 | 3.45 | 3.39 | 3.35 | 1.15 | 45.8% |
| Fluoresc. | 519-760 | 4.33 | 4.25 | 4.19 | 4.13 | 1.35 | 53.7% |
| total | 400-760 | 7.861847 | 7.697121 | 7.575976 | 7.4794 | 2.50 | 99.4% |
| % fluorescence | | 55.0% | 55.2% | 55.3% | 55.2% | 0.54 | 54.0% |
| purple | (400)-450 | 3.0825 | 2.9951 | 2.9309 | 2.8833 | 1.01 | 40.2% |
| Blue | 450-500 | 0.4529 | 0.4523 | 0.4582 | 0.4662 | 0.14 | 5.5% |
| Green | 500-570 | 0.3313 | 0.3202 | 0.3118 | 0.3050 | 0.11 | 4.4% |
| Yellow | 570-591 | 1.4610 | 1.4288 | 1.4024 | 1.3776 | 0.46 | 18.4% |
| Orange | 591-610 | 1.1464 | 1.1263 | 1.1076 | 1.0910 | 0.36 | 14.2% |
| Red | 610-760 | 1.4325 | 1.4186 | 1.4085 | 1.3993 | 0.43 | 17.1% |
| total | (400-700) | 7.91 | 7.74 | 7.62 | 7.52 | 2.52 | 100.0% |

TABLE 1B

| | | mW/cm2 at 5 cm | | | | | | |
|---|---|---|---|---|---|---|---|---|
| pHEMA transparent, secdisk 2.0 | | 0 | 0.5 min | 1 min | 1.5 min | 2 min | 2.5 min | 3 min |
| Lamp | 400-518 | 15.93 | 15.92 | 15.85 | 15.78 | 15.49 | 15.37 | 15.25 |
| Fluoresc. | 519-760 | 1.81 | 1.84 | 1.82 | 1.78 | 1.70 | 1.66 | 1.63 |
| total | 400-760 | 17.73536 | 17.76091 | 17.67241 | 17.56012 | 17.19044 | 17.03573 | 16.87824 |
| % fluorescence | | 10.2% | 10.4% | 10.3% | 10.2% | 9.9% | 9.8% | 9.6% |
| purple | (400)-450 | 10.0240 | 9.8565 | 9.7204 | 9.5544 | 9.1695 | 9.0147 | 8.8467 |
| Blue | 450-500 | 5.5175 | 5.6830 | 5.7647 | 5.8590 | 5.9756 | 6.0153 | 6.0629 |
| Green | 500-570 | 1.5844 | 1.5748 | 1.5478 | 1.5166 | 1.4431 | 1.4105 | 1.3805 |
| Yellow | 570-591 | 0.2619 | 0.2660 | 0.2616 | 0.2589 | 0.2471 | 0.2417 | 0.2390 |
| Orange | 591-610 | 0.1627 | 0.1674 | 0.1654 | 0.1623 | 0.1561 | 0.1536 | 0.1499 |
| Red | 610-760 | 0.1917 | 0.2200 | 0.2193 | 0.2152 | 0.2050 | 0.2050 | 0.2052 |
| total | (400-700) | 17.74 | 17.77 | 17.68 | 17.57 | 17.20 | 17.04 | 16.88 |

| | | mW/cm2 at 5 cm | | | | | |
|---|---|---|---|---|---|---|---|
| pHEMA transparent, secdisk 2.0 | | 3.5 min | 4 min | 4.5 min | 5 min | J/cm2 | |
| Lamp | 400-518 | 15.14 | 15.02 | 14.91 | 14.91 | 4.64 | 90.1% |
| Fluoresc. | 519-760 | 1.58 | 1.54 | 1.48 | 1.48 | 0.51 | 9.8% |
| total | 400-760 | 16.72025 | 16.55176 | 16.39374 | 16.39511 | 5.14 | 100.0% |
| % fluorescence | | 9.5% | 9.3% | 9.0% | 9.1% | 0.10 | 9.8% |
| purple | (400)-450 | 8.7059 | 8.5500 | 8.4174 | 8.4294 | 2.76 | 53.5% |
| Blue | 450-500 | 6.1003 | 6.1392 | 6.1707 | 6.1605 | 1.78 | 34.6% |
| Green | 500-570 | 1.3430 | 1.3102 | 1.2728 | 1.2688 | 0.43 | 8.4% |
| Yellow | 570-591 | 0.2318 | 0.2251 | 0.2182 | 0.2154 | 0.07 | 1.4% |
| Orange | 591-610 | 0.1477 | 0.1425 | 0.1416 | 0.1391 | 0.05 | 0.9% |
| Red | 610-760 | 0.1979 | 0.1904 | 0.1787 | 0.1875 | 0.05 | 1.2% |
| total | (400-700) | 16.73 | 16.56 | 16.40 | 16.40 | 5.15 | 100.0% |

TABLE 1C

| | | mW/cm2 at 5 cm | | | | | | |
|---|---|---|---|---|---|---|---|---|
| pHEMA, secdisk SABLE, 2.3 mm, F [symbol] | | 0 | 0.5 min | 1 min | 1.5 min | 2 min | 2.5 min | 3 min |
| lamp | 400-518 | 12.03 | 12.08 | 12.13 | 12.14 | 12.12 | 12.08 | 12.06 |
| Fluoresc. | 519-760 | 12.29 | 12.11 | 11.81 | 11.66 | 11.46 | 11.26 | 11.10 |
| total | 400-760 | 24.32164 | 24.18741 | 23.94023 | 23.80006 | 23.58084 | 23.34528 | 23.16125 |
| % fluorescence | | 50.5% | 50.1% | 49.3% | 49.0% | 48.6% | 48.2% | 47.9% |
| purple | (400)-450 | 8.3959 | 8.2868 | 8.2218 | 8.1749 | 8.1014 | 8.0206 | 7.9541 |
| Blue | 450-500 | 3.5779 | 3.7245 | 3.8376 | 3.8944 | 3.9523 | 3.9983 | 4.0408 |
| Green | 500-570 | 1.7594 | 1.6903 | 1.6006 | 1.5587 | 1.5115 | 1.4630 | 1.4294 |
| Yellow | 570-591 | 3.9329 | 3.8400 | 3.7147 | 3.6517 | 3.5752 | 3.4981 | 3.4293 |
| Orange | 591-610 | 2.9743 | 2.9482 | 2.9034 | 2.8730 | 2.8395 | 2.7978 | 2.7559 |
| Red | 610-760 | 3.7967 | 3.8128 | 3.7767 | 3.7604 | 3.7134 | 3.6785 | 3.6613 |
| total | (400-700) | 24.44 | 24.30 | 24.05 | 23.91 | 23.69 | 23.46 | 23.27 |

| | | mW/cm2 at 5 cm | | | | | |
|---|---|---|---|---|---|---|---|
| pHEMA, secdisk SABLE, 2.3 mm, F [symbol] | | 3.5 min | 4 min | 4.5 min | 5 min | J/cm2 | |
| lamp | 400-518 | 12.04 | 12.04 | 12.02 | 12.04 | 3.62 | 51.2% |
| Fluoresc. | 519-760 | 10.95 | 10.82 | 10.65 | 10.56 | 3.42 | 48.4% |
| total | 400-760 | 22.98993 | 22.85589 | 22.66212 | 22.60164 | 7.05 | 99.5% |
| % fluorescence | | 47.6% | 47.3% | 47.0% | 46.7% | 0.49 | 48.6% |
| purple | (400)-450 | 7.8954 | 7.8462 | 7.7835 | 7.7551 | 2.42 | 34.2% |
| Blue | 450-500 | 4.0794 | 4.1285 | 4.1706 | 4.2188 | 1.18 | 16.7% |
| Green | 500-570 | 1.4007 | 1.3819 | 1.3535 | 1.3470 | 0.45 | 6.4% |
| Yellow | 570-591 | 3.3643 | 3.3049 | 3.2505 | 3.2169 | 1.07 | 15.1% |
| Orange | 591-610 | 2.7159 | 2.6745 | 2.6399 | 2.6151 | 0.84 | 11.9% |
| Red | 610-760 | 3.6423 | 3.6268 | 3.5696 | 3.5534 | 1.11 | 15.7% |
| total | (400-700) | 23.10 | 22.96 | 22.77 | 22.71 | 7.08 | 100.0% |

[symbol]

indicates data missing or illegible when filed

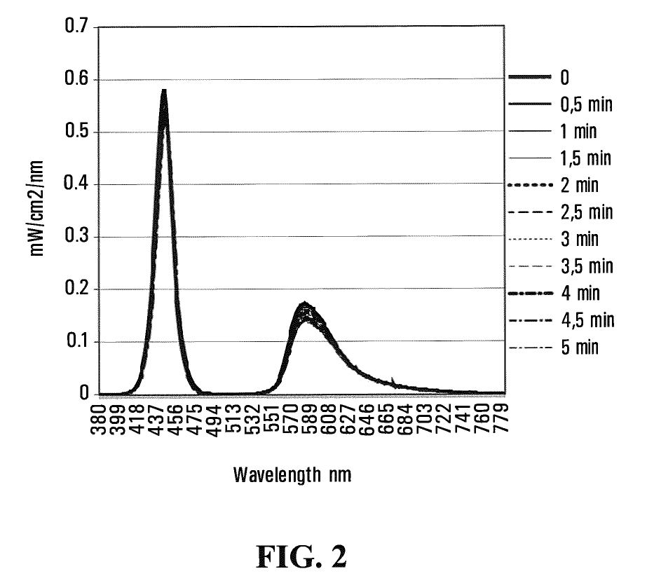

FIG. 2 shows the light emission spectra of a 2.3 mm thick block opaque pHEMA matrix for an illumination period of 5 minutes. Table 2 below shows the emission data corresponding to FIG. 2.

TABLE 2

| | | mW/cm2 at 5 cm | | | | | | |
|---|---|---|---|---|---|---|---|---|
| pHEMA BLOCK OPAQUE 2.3 mm [symbol] | | 0 | 0.5 min | 1 min | 1.5 min | 2 min | 2.5 min | 3 min |
| Lamp | 400-518 | 12.67 | 12.50 | 12.33 | 12.17 | 12.05 | 11.95 | 11.95 |
| Fluoresc. | 519-760 | 10.50 | 10.36 | 10.18 | 10.00 | 9.86 | 9.67 | 9.55 |
| total | 400-760 | 23.17033 | 22.85598 | 22.51655 | 22.1691 | 21.91052 | 21.62494 | 21.49502 |
| % fluorescence | | 45.3% | 45.3% | 45.2% | 45.1% | 45.0% | 44.7% | 44.4% |
| purple | (400)-450 | 9.6609 | 9.3930 | 9.1876 | 8.9848 | 8.8207 | 8.6601 | 8.5597 |
| Blue | 450-500 | 3.0077 | 3.1001 | 3.1359 | 3.1723 | 3.2165 | 3.2817 | 3.3729 |
| Green | 500-570 | 1.1351 | 1.0867 | 1.0359 | 0.9951 | 0.9670 | 0.9390 | 0.9351 |
| Yellow | 570-591 | 3.3940 | 3.3090 | 3.2246 | 3.1447 | 3.0744 | 2.9986 | 2.9331 |
| Orange | 591-610 | 2.7124 | 2.6938 | 2.6656 | 2.6308 | 2.5952 | 2.5473 | 2.5010 |
| Red | 610-760 | 3.3656 | 3.3789 | 3.3713 | 3.3452 | 3.3392 | 3.2999 | 1.2923 |
| total | (400-700) | 23.28 | 22.96 | 22.52 | 22.27 | 22.01 | 21.73 | 21.59 |

| | | mW/cm2 at 5 cm | | | | | |
|---|---|---|---|---|---|---|---|
| pHEMA BLOCK OPAQUE 2.3 mm [symbol] | | 3.5 min | 4 min | 4.5 min | 5 min | J/cm2 | |
| Lamp | 400-518 | 12.01 | 12.10 | 12.30 | 12.57 | 3.66 | 55.3% |
| Fluoresc. | 519-760 | 9.40 | 9.18 | 9.04 | 8.90 | 2.93 | 44.3% |
| total | 400-760 | 21.40408 | 21.28549 | 21.33923 | 21.46927 | 6.59 | 99.5% |
| % fluorescence | | 43.9% | 43.1% | 42.4% | 41.4% | 0.44 | 44.5% |
| purple | (400)-450 | 8.5006 | 8.4713 | 8.4902 | 8.5511 | 2.66 | 40.2% |
| Blue | 450-500 | 3.4893 | 3.6177 | 3.7949 | 4.0056 | 1.00 | 15.0% |
| Green | 500-570 | 0.9327 | 0.9183 | 0.9311 | 0.9473 | 0.30 | 4.5% |
| Yellow | 570-591 | 2.8667 | 2.7968 | 2.7383 | 2.6878 | 0.91 | 13.8% |
| Orange | 591-610 | 2.4487 | 2.3892 | 2.3322 | 2.2733 | 0.77 | 11.6% |
| Red | 610-760 | 3.2637 | 3.1876 | 3.1462 | 3.0956 | 0.99 | 14.9% |
| total | (400-700) | 21.50 | 21.38 | 21.43 | 21.56 | 6.62 | 100.0% |

[symbol]

indicates data missing or illegible when filed

### Example 2

### Preparation of a Hydrogel of pHEMA Incorporating Fluorescein

A hydrogel of polymerized HEMA incorporating Fluorescein was prepared by mixing together the following materials:

1) Pure water-Hyclone (70 ml) #SH30529.02, from Thermo Scientific;
2) Benzoyl peroxide (100 mg) (BPO) #517909, from Sigma Aldrich Co. USA, which was used as initiator;
3) Fluorescein (50 mg) D&C Yellow 8, #2012-27110, from Spectra Colors Corporation;
4) 2-Hydroxyethyl methacrylate (30 ml) (HEMA) #477028, from Sigma Aldrich Co. USA; and
5) Ethylene glycol dimethacrylate (500 μl) (EGDMA) #335681, from Sigma Aldrich Co. USA, which was used as cross-linker.

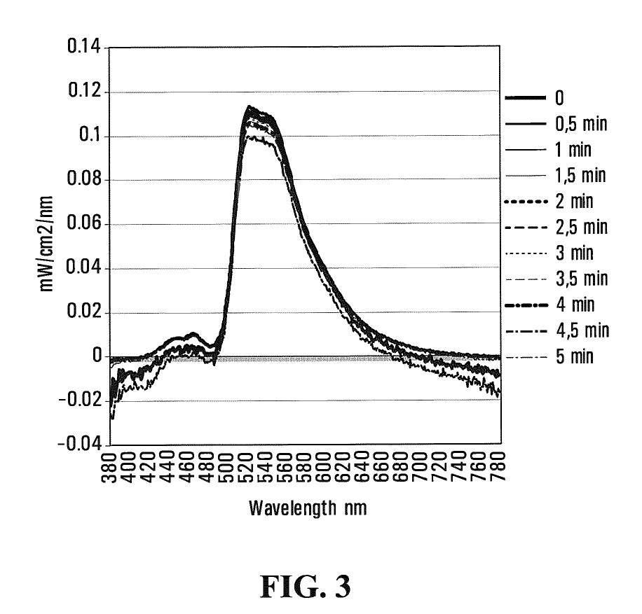

Polymerization was carried out in a nitrogen atmosphere in 5 hours at 70° C. Once polymerized, the hydrogel was kept in a sealed container to avoid dehydration. FIG. 3 shows the light emission spectra of a 2.3 mm thick pHEMA hydrogel during 5 minutes of illumination. Table 3 below shows the emission data corresponding to FIG. 3.

TABLE 3

| | | mw/cm2 at 5 cm | | | | | | |
|---|---|---|---|---|---|---|---|---|
| 2.30 mm, pHEMA, hydrogel (F50) | | 0 | 0.5 min | 1 min | 1.5 min | 2 min | 2.5 min | 3 min |
| Lamp | 400-518 | 1.31 | 1.84 | 1.86 | 1.85 | 1.82 | 1.81 | 1.79 |
| Fluoresc. | 519-760 | 7.86 | 8.66 | 8.63 | 8.54 | 8.45 | 8.39 | 8.31 |
| total | 400-760 | 9.164657 | 10.49897 | 10.48605 | 10.33797 | 10.27257 | 10.20414 | 10.1088 |
| % fluorescence | | 85.7% | 82.5% | 82.3% | 82.2% | 82.3% | 82.3% | 82.3% |
| purple | (400)-450 | −0.1417 | 0.1446 | 0.1554 | 0.1538 | 0.1501 | 0.1510 | 0.1486 |
| Blue | 450-500 | 0.2318 | 0.4363 | 0.4478 | 0.4502 | 0.4463 | 0.4485 | 0.4482 |
| Green | 500-570 | 6.4839 | 6.6430 | 6.6006 | 6.5365 | 6.4547 | 6.4009 | 6.3427 |
| Yellow | 570-591 | 1.2232 | 1.2711 | 1.2645 | 1.2532 | 1.2407 | 1.2326 | 1.2231 |
| Orange | 591-610 | 0.7494 | 0.8045 | 0.8026 | 0.7956 | 0.7877 | 0.7827 | 0.7759 |
| Red | 610-760 | 0.6482 | 1.2322 | 1.2477 | 1.2310 | 1.2250 | 1.2201 | 1.2019 |
| total | (400-700) | 9.19 | 10.53 | 10.52 | 10.42 | 10.30 | 10.24 | 10.14 |

| | | mw/cm2 at 5 cm | | | | | |
|---|---|---|---|---|---|---|---|
| 2.30 mm, pHEMA, hydrogel (F50) | | 3.5 min | 4 min | 4.5 min | 5 min | J/cm2 | |
| Lamp | 400-518 | 1.78 | 1.77 | 0.74 | 1.69 | 0.50 | 16.8% |
| Fluoresc. | 519-760 | 8.22 | 8.16 | 6.42 | 7.95 | 2.45 | 82.9% |
| total | 400-760 | 9.996379 | 9.927272 | 7.16039 | 9.631762 | 2.95 | 99.7% |
| % fluorescence | | 82.2% | 82.2% | 89.7% | 82.5% | 0.83 | 83.1% |
| purple | (400)-450 | 0.1427 | 0.1421 | −0.4192 | 0.0984 | 0.02 | 0.6% |
| Blue | 450-500 | 0.4497 | 0.4498 | 0.0754 | 0.4292 | 0.12 | 3.9% |
| Green | 500-570 | 6.2742 | 6.2334 | 5.8257 | 6.1465 | 1.91 | 64.8% |
| Yellow | 570-591 | 1.2097 | 1.2028 | 1.0853 | 1.1825 | 0.37 | 12.4% |
| Orange | 591-610 | 0.7680 | 0.7631 | 0.6497 | 0.7476 | 0.23 | 7.8% |
| Red | 610-760 | 1.1833 | 1.1669 | −0.0312 | 1.0578 | 0.31 | 10.5% |
| total | (400-700) | 10.03 | 9.96 | 7.19 | 9.66 | 2.96 | 100.0% |

### Example 3

### Preparation of Microspheres of Polymerized HEMA Incorporating Eosin Y in Silicon Matrix

Microspheres of polymerized HEMA incorporating Eosin Y were prepared by mixing together the materials of in the order given below in vegetable oil and in a silicone matrix (1:15 (v/v)), while stirring at 600 rpm.

1) Pure water-Hyclone (30 ml) #SH30529.02, from Thermo Scientific;
2) Benzoyl peroxide (100 mg) (BPO) #51790, from Sigma Aldrich Co. USA, which was used as initiator;
3) Eosin Y (100 mg) D&C Red22 #2012-27447, from Spectra Colors Corporation;
4) 2-Hydroxyethyl methacrylate (70 ml) (HEMA) #477028, from Sigma Aldrich Co. USA; and
5) Ethylene glycol dimethacrylate (500 μl) (EGDMA) #335681, from Sigma Aldrich Co. USA, which was used as cross-linker.

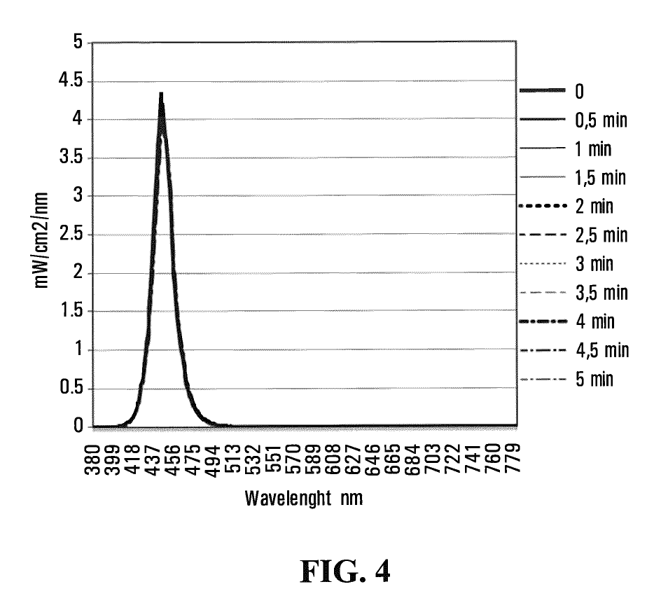

Polymerization was conducted in a nitrogen atmosphere in 4 hours at 60° C. Toluene was obtained from Fisher #062843. Ethyl ether was obtained from Fisher #124158 and was used to remove any traces of unreacted monomers. The particles were rinsed twice with Toluene. Then these particles were rinsed with ethyl ether to remove any traces of unreacted monomers and organic residue. FIG. 4 shows the light emission spectra of a 2.0 mm thick silicone, pHEMA microspheres during 5 minutes of illumination, according to an embodiment of the present disclosure. Table 4 below shows the emission data corresponding to FIG. 4.

TABLE 4

| | | mW/cm2 at 5 cm | | | | | | |
|---|---|---|---|---|---|---|---|---|
| 2.00 mm silicon, pHEMA microsp [symbol] | | 0 | 0.5 min | 1 min | 1.5 min | 2 min | 2.5 min | 3 min |
| Lamp | 400-518 | 102.21 | 101.56 | 101.57 | 101.27 | 100.94 | 100.56 | 100.34 |
| Fluoresc. | 519-760 | 0.41 | 0.44 | 0.41 | 0.39 | 0.36 | 0.33 | 0.35 |
| total | 400-760 | 102.6241 | 101.9988 | 101.9772 | 101.6593 | 101.3035 | 100.8931 | 100.691 |
| % fluorescence | | 0.4% | 0.4% | 0.4% | 0.4% | 0.4% | 0.3% | 0.3% |
| purple | (400)-450 | 65.0353 | 63.6121 | 62.7861 | 62.2876 | 61.6326 | 61.0283 | 60.5961 |
| Blue | 450-500 | 37.0213 | 37.7835 | 38.6046 | 38.8072 | 39.1243 | 39.3617 | 39.5573 |
| Green | 500-570 | 0.3902 | 0.4253 | 0.4152 | 0.4153 | 0.3878 | 0.3839 | 0.3942 |
| Yellow | 570-591 | 0.1078 | 0.1072 | 0.1042 | 0.0917 | 0.0918 | 0.0765 | 0.0855 |
| Orange | 591-610 | 0.0559 | 0.0564 | 0.0544 | 0.0475 | 0.0520 | 0.0353 | 0.0461 |
| Red | 610-760 | 0.0153 | 0.0162 | 0.0143 | 0.0115 | 0.0168 | 0.0086 | 0.0133 |
| total | (400-700) | 102.63 | 102.00 | 101.98 | 101.66 | 101.31 | 100.89 | 100.69 |

| | | mW/cm2 at 5 cm | | | | | |
|---|---|---|---|---|---|---|---|
| 2.00 mm silicon, pHEMA microsp [symbol] | | 3.5 min | 4 min | 4.5 min | 5 min | J/cm2 | |
| Lamp | 400-518 | 100.08 | 99.77 | 99.70 | 99.51 | 30.24 | 99.6% |
| Fluoresc. | 519-760 | 0.34 | 0.32 | 0.33 | 0.33 | 0.11 | 0.4% |
| total | 400-760 | 100.4174 | 100.09 | 100.0314 | 99.83918 | 30.35 | 100.0% |
| % fluorescence | | 0.3% | 0.3% | 0.3% | 0.3% | 0.00 | 0.4% |
| purple | (400)-450 | 60.1337 | 59.6929 | 59.4911 | 59.1823 | 18.49 | 60.9% |
| Blue | 450-500 | 39.7597 | 39.8845 | 40.0303 | 40.1380 | 11.70 | 38.5% |
| Green | 500-570 | 0.4005 | 0.3936 | 0.3837 | 0.3988 | 0.12 | 0.4% |
| Yellow | 570-591 | 0.0712 | 0.0700 | 0.0745 | 0.0682 | 0.03 | 0.1% |
| Orange | 591-610 | 0.0412 | 0.0373 | 0.0403 | 0.0391 | 0.01 | 0.0% |
| Red | 610-760 | 0.0126 | 0.0129 | 0.0129 | 0.0142 | 0.00 | 0.0% |
| total | (400-700) | 100.42 | 100.09 | 100.03 | 99.84 | 30.35 | 100.0% |

[symbol]

indicates data missing or illegible when filed

### Example 4

### Evaluation of Anti-Inflammatory Properties of Emissive Polymeric Matrices Illuminated with Blue Light (Using Thera™ Lamp)

HaCaT human keratinocytes were used as in vitro model to study the effect of the visible blue light in combination with the emissive polymeric matrices as defined herein on the secretion of the pro-inflammatory cytokines such as IL6 and IL8. The excessive, uncontrolled inflammation is detrimental to the host and impairs wound healing process. Therefore the purpose of this study is to demonstrate that blue light in combination with the emissive polymeric matrices is able to down-regulate the production of pro-inflammatory cytokines and improve the healing process. A non-toxic concentration of IFNγ was used to modulate the secretion of IL6 and IL8 by HaCaT cells. Dexamethasone (final concentration of 5 μM) was used as a positive control (strong inhibitor of pro-inflammatory cytokine production). HaCaT cells were illuminated for 90 sec with THER™ lamp at the distance of 5 cm in the combination with the following matrices:
1. Matrix 1: pHEMA Block opaque 4 mm (50 mg Eosin/50 mg Fluorescein);
2. Matrix 2: pHEMA Bock opaque 2.3 mm (50 mg Eosin/50 mg Fluorescein);
3. Matrix 3: pHEMA Hydrogel (50 mg Eosin/50 mg Fluorescein); and
4. Matrix 4: pHEMA Hydrogel (50 mg Fluorescein).

### Matrix 1 and Matrix 2:

### Materials

Pure water Hyclone (30 ml) #SH30529.02 from Thermo Scientific;
Benzoyl peroxide (100 mg) (BPO) #517909 from Sigma Aldrich Co. USA, used as the initiator;
Eosin Y (50 mg) D&C Red22 #2012-27447 from Spectra Colors Corporation;
Fluorescein (50 mg) D&C Yellow 8, #2012-27110 from Spectra Colors Corporation;
2-Hydroxyethyl methacrylate (70 ml) (HEMA) #477028, from Sigma Aldrich Co. USA;
Ethylene glycol dimethacrylate (500 μl) (EGDMA) #335681, from Sigma Aldrich Co. USA, used as the cross-linker.

### Method

The materials were added and mixed in the order given above. Eosin Y and Fluorescein were added to liquid monomer pre-polymerization. Polymerization was conducted in a nitrogen atmosphere for 5 hours at 70° C.

### Matrix 3:

### Materials

Pure water Hyclone (70 ml) #SH30529.02 from Thermo Scientific;
Benzoyl peroxide (100 mg) (BPO) #517909 from Sigma Aldrich Co. USA, used as the initiator;
Eosin Y (50 mg) D&C Red22 #2012-27447 from Spectra Colors Corporation;
Fluorescein (50 mg) D&C Yellow 8, #2012-27110 from Spectra Colors Corporation;
2-Hydroxyethyl methacrylate (30 ml) (HEMA) #477028, from Sigma Aldrich Co. USA;
Ethylene glycol dimethacrylate (500 μl) (EGDMA) #335681, from Sigma Aldrich Co. USA, used as the cross-linker.

### Method

The materials were added and mixed in the order given above. Eosin Y and Fluorescein were added to liquid monomer pre-polymerization. Polymerization was conducted in a nitrogen atmosphere for 5 hours at 70° C.

### Matrix 4:

Pure water Hyclone (70 ml) #SH30529.02 from Thermo Scientific;
Benzoyl peroxide (100 mg) (BPO) #517909 from Sigma Aldrich Co. USA, used as the initiator;
Fluorescein (50 mg) D&C Yellow 8, #2012-27110 from Spectra Colors Corporation;
2-Hydroxyethyl methacrylate (30 ml) (HEMA) #477028, from Sigma Aldrich Co. USA;
Ethylene glycol dimethacrylate (500 μl) (EGDMA) #335681, from Sigma Aldrich Co. USA, used as the cross-linker.

### Method

The materials were added and mixed in the order given above. Eosin Y and Fluorescein were added to liquid monomer pre-polymerization. Polymerization was conducted in a nitrogen atmosphere for 5 hours at 70° C. The photonic evaluation was performed for each of the above mentioned matrices using a THERA™ lamp. The matrices were illuminated for 5 min total at a distance of 5 cm from the light source. The measurements obtained are shown below in the following tables, presented in sequential register where Table 5 corresponds to Matrix 1, Table 6 corresponds to Matrix 2, Table 7 corresponds to Matrix 3 and Table 8 corresponds to Matrix 4.

Cytokine quantification was performed by using cytokine ELISA (DuoSet ELISA development kit from R&D Systems). For each experiment, the XTT assay was performed to normalize the quantity of cytokine secreted to the cell viability. All samples were screened in quadruplets. The results of the biological effect of tested matrices in the combination with visible blue light are summarized in the Table 9 below:

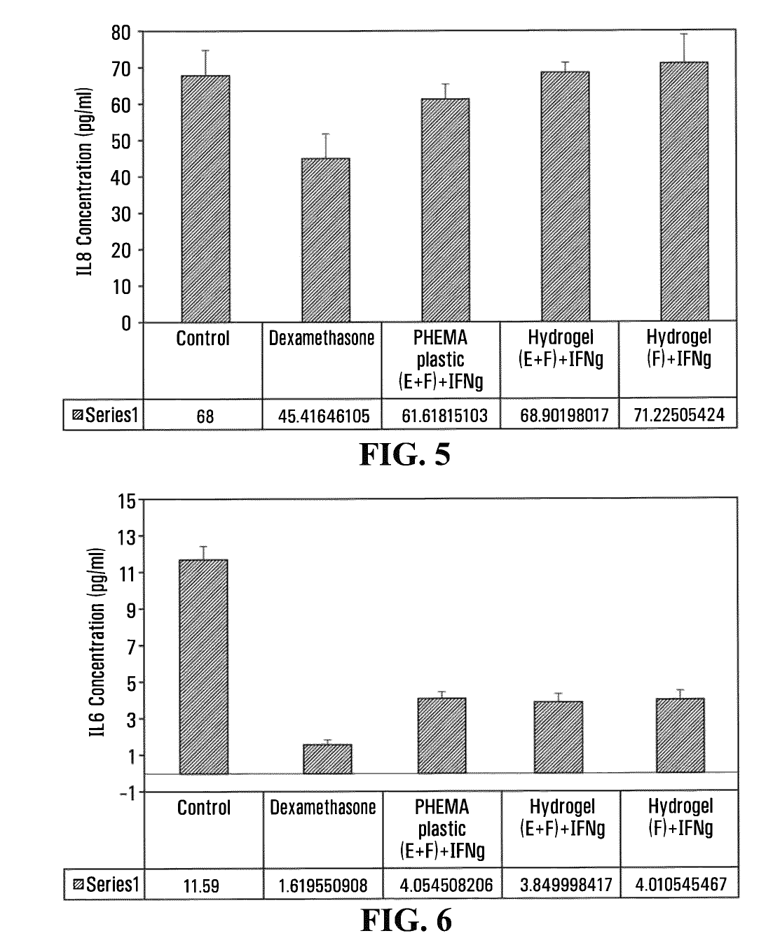

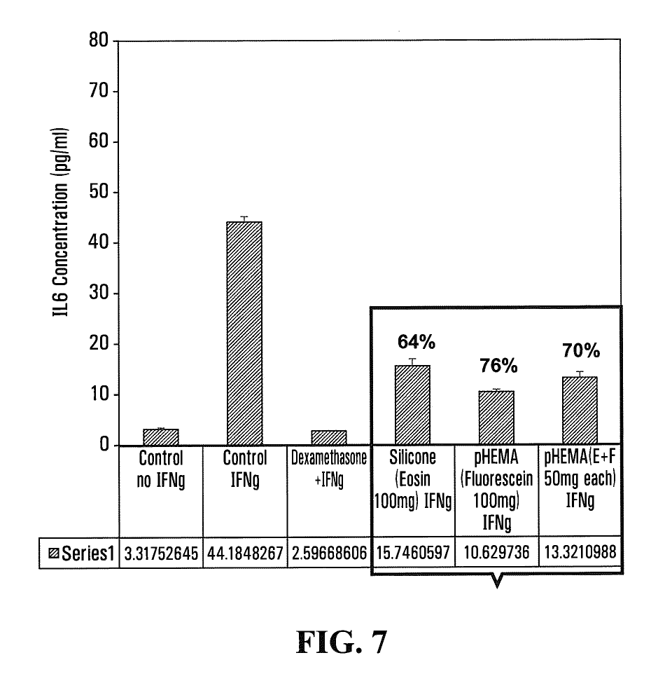

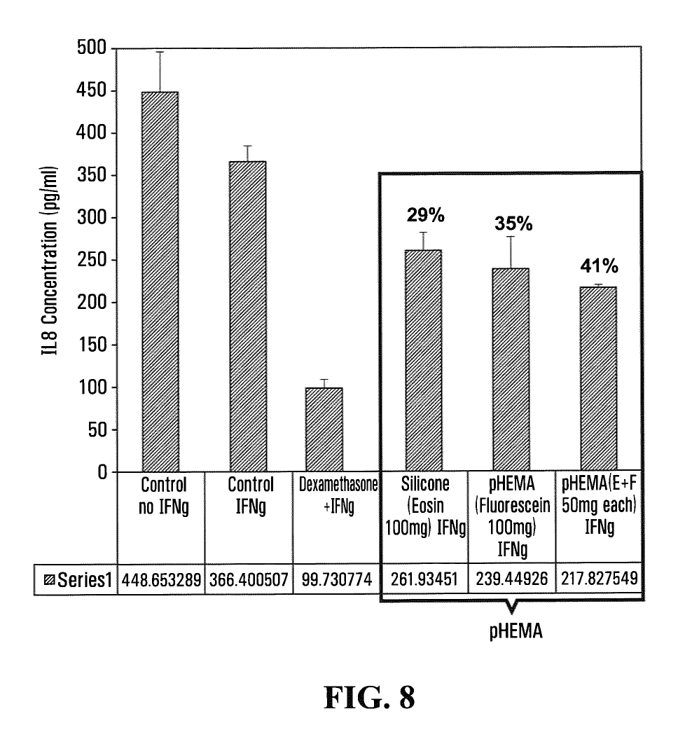

The comparison of matrices which blocked blue light (i.e. pHEMA hydrogel) with the matrices which allow blue light penetration revealed that the ones that block blue light (i.e. pHEMA hydrogel) are effective in downregulating both (IL6 and IL8) pro-inflammatory cytokines secretion. All matrices tested were effective in downregulating IL6. FIGS. 5, 6, 7 and 8 illustrate the IL6 and IL8 secretion by IFNγ stimulated HaCaT cells exposed to the indicated emissive polymeric matrices of the present disclosure.

### Example 5

### Testing Bleaching of Eosin Y within a pHEMA Matrix

The photobleaching of Eosin Y within a pHEMA matrix was tested after exposure of the pHEMA matrix to freezing temperatures. The data are reported in Table 10 below. The matrix was prepared according to the particulars below:

### Materials

Pure water Hyclone (30 ml) #SH30529.02 from Thermo Scientific;
Benzoyl peroxide (100 mg) (BPO) #517909 from Sigma Aldrich Co. USA, used as the initiator;
Eosin Y (50 mg) (for #3) and (100 mg) (for #1, #2, #4) D&C Red22 #2012-27447 from Spectra Colors Corporation;
2-Hydroxyethyl methacrylate (70 ml) (HEMA) #477028, from Sigma Aldrich Co. USA;
Ethylene glycol dimethacrylate (500 μl) (EGDMA) #335681, from Sigma Aldrich Co. USA, used as the cross-linker.

### Method

The materials were added and mixed in the order given above. Eosin Y was added to liquid monomer pre-polymerization. Polymerization was conducted in a nitrogen atmosphere for 5 hours at 70° C.

### Example 6

### Increase in pHEMA (Disk) Temperature with/without Chromophores

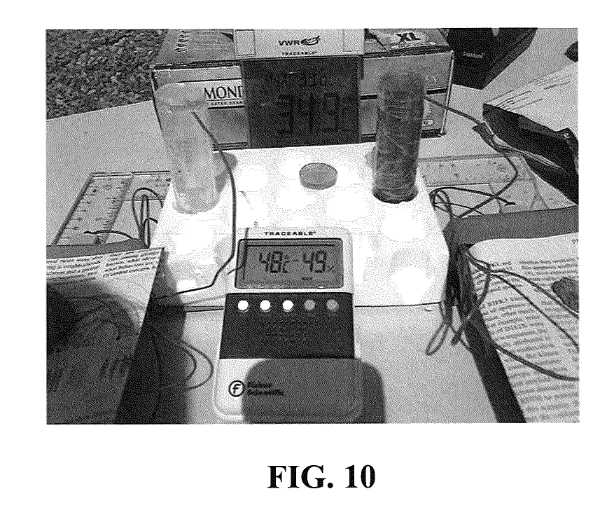

Temperature of a pHEMA matrix according to an embodiment of the present disclosure comprising chromophores or without chromophores was measured after exposure to sunlight for the amount of time indicated in Table 11 below. The experiment was carried out according to the experimental set-up depicted in FIG. 10.

TABLE 11

| | | | |
|---|---|---|---|
| START | 5 minutes | 10 minutes | 15 minutes |

Temperature of pHEMA disk, 2.0 mm
(Eosin 50 mg + Fluorescein 50 mg)

| | | | |
|---|---|---|---|
| a1 = 26.9° C. | a2 = 41.9° C. | a3 = 47.3° C. | a4 = 48.7° C. |

Temperature of pHEMA disk, 2.0 mm (no chromophore)

| | | | |
|---|---|---|---|
| b1 = 21.3° C. | b2 = 28.0° C. | b3 = 30.9° C. | b4 = 32.3° C. |
| a1-b1 = 5.6° C. | a2-b2 = 13.9° C. | a3-b3 = 16.4° C. | a4-b4 = 16.4° C. |

Thera Lamp, no ID
0 min to 5 mins to 10 mins to 15 mins “LIGHT NON STOP” = 30 mins Total Distance lamp to disk = 5 cm
(a1 . . . a4) = temperature for pHEMA disk with chromophore (Eosin Y + Fluorescein/50 mg each)
(b1 . . . b4) = temperature for pHEMA disk without chromophore

### Example 7

### Increase in H₂O Temperature Using pHEMA(Disk) with/without Chromophores

Temperature of a H₂O surrounding a pHEMA matrix according to an embodiment of the disclosure comprising chromophores or without chromophores as outlined in Example 6 was measured after exposure of the pHEMA matrix to sunlight according to the experimental set-up depicted in FIG. 10. The pHEMA containers (+1-chromophores) were filled with water (8 ml of water); covered with a lid of the same pHEMA composition; and were exposed to sunlight for the period of time indicated in Table 12 below. Temperature of the water in the pHEMA containers was measured and data is presented in Tables 12, 13 and 14 below.

TABLE 12
Water temperature in a pHEMA container (8 ml)
Assay #1

| | | | | |
|---|---|---|---|---|
| 10 h 00 | 10 h 30 | 11 h 00 | 11 h 30 | 12 h 00 |

Water temperature (° C.) in pHEMA container comprising
chromophores Eosin Y (50 mg) + Fluorescein
(50 mg) per 100 g of polymer

| | | | | |
|---|---|---|---|---|
| 24.6 | 37.1 | 41.2 | 39.9 | 41.0 |

Water temperature (° C.) in pHEMA container without chromophores

| | | | | |
|---|---|---|---|---|
| 24.7 | 34.0 | 37.4 | 36.7 | 36.0 |

Ambient Temperature (° C.)

| | | | | |
|---|---|---|---|---|
| — | — | 35.1 | 34.6 | 34.8 |

E50 + F50 = 50 mg Eosin Y + 50 mg Fluorescein/100 g polymer

TABLE 13
Water temperature in a pHEMA container (8 ml)
Assay #2 - Container made using fresh chromophores
Water temperature (° C.) in pHEMA container comprising
chromophores Eosin Y (50 mg) + Fluorescein
(50 mg) per 100 g of polymer

| 10 h 30 | 11 h 00 | 11 h 30 | 12 h 00 | 12 h 30 |
|---|---|---|---|---|
| 24.7 | 36.8 | 39.9 | 41.0 | 40.2 |
| 10 h 00 | 10 h 30 | 11 h 00 | 11 h 30 | 12 h 00 |

Water temperature (° C.) in pHEMA container without chromophores

| | | | | |
|---|---|---|---|---|
| 23.0 | 33.0 | 36.2 | 37.0 | 37.2 |

Ambient Temperature (° C.)

| | | | | |
|---|---|---|---|---|
| 25.4 | 38.7 | 39.2 | 41.1 | 40.7 |

E50 + F50 = 50 mg Eosin Y + 50 mg Fluorescein/100 g polymer

TABLE 14
Water temperature in a pHEMA container (8 ml)
Assay #3
Water temperature (° C.) in pHEMA container without chromophores

| 10 h 30 | 11 h 00 | 11 h 30 | 12 h 00 | 12 h 30 |
|---|---|---|---|---|
| 24.2 | 29.6 | 31.7 | 32.2 | 31.2 |
| 10 h 00 | 10 h 30 | 11 h 00 | 11 h 30 | 12 h 00 |

Water temperature (° C.) in pHEMA container comprising
chromophores E50 + F50 - Container
made using fresh chromophores

| | | | | |
|---|---|---|---|---|
| 23.6 | 31.5 | 34.3 | 34.3 | 33.7 |

Water temperature (° C.) in pHEMA container comprising
chromophores E50 + F50 - Reused
Container from Assay #1

| | | | | |
|---|---|---|---|---|
| 23.1 | 30 | 32.8 | 32.7 | 36.5 |

Ambient Temperature (° C.)

| | | | | |
|---|---|---|---|---|
| 24.5 | 31.5 | 30.1 | 31.5 | 35.5 |

E50 + F50 = 50 mg Eosin Y + 50 mg Fluorescein/100 g polymer

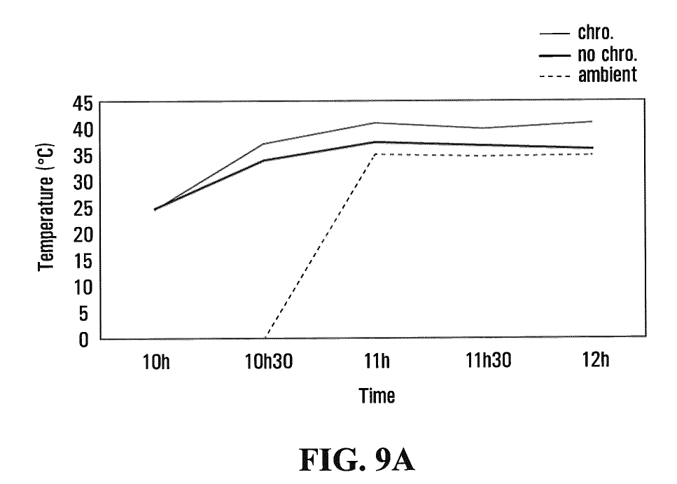

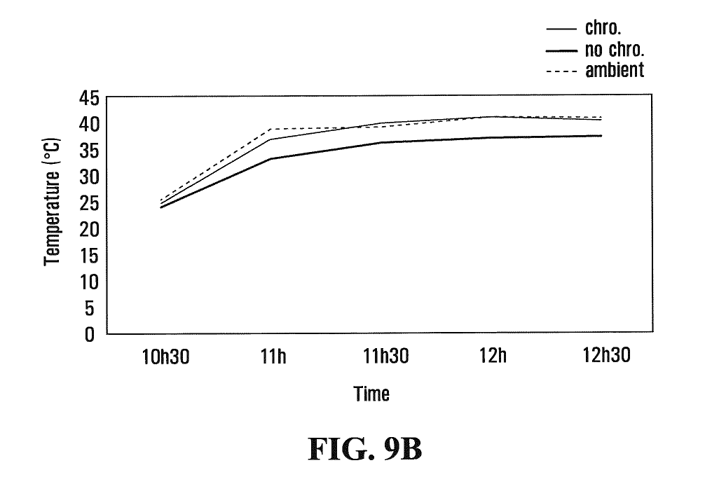

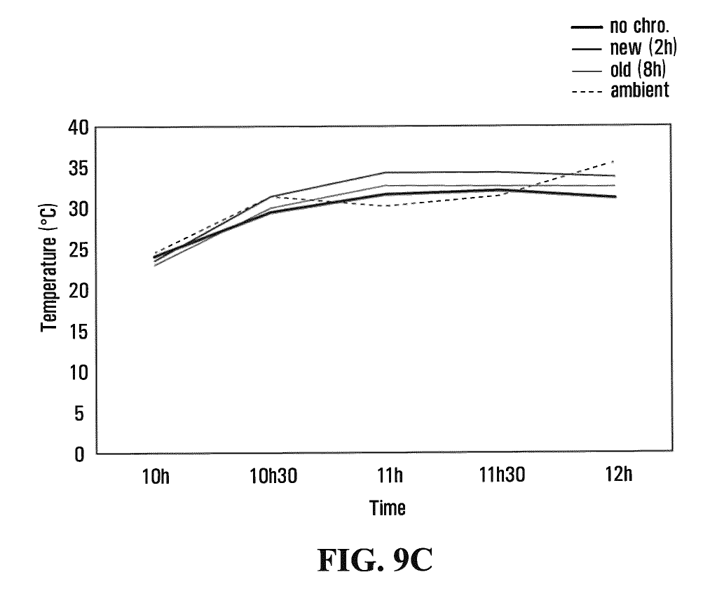

As shown in FIGS. 9A, 9B and 9C, the temperature of the water in the pHEMA container with the chromophores is higher than the temperature of the water in the pHEMA container without chromophores, indicating that generation of thermal radiation by the pHEMA matrix is greater in the presence of chromophores. FIGS. 9A, 9B and 9C also show that the temperature of the water in the pHEMA container without chromophore was similar to the ambient temperature. Upon exposure to sunlight, the pHEMA container with the chromophore was also able to emit fluorescence whereas the pHEMA container without chromophore was not (data not shown). The experimental data obtained in this Example 7 demonstrates that upon exposure to sunlight, the pHEMA matrix comprising the chromophore(s) generated radiation that was diffused to the surrounding environment of the matrix and which caused the temperature of the water to increase. This shows that a pHEMA matrix comprising the chromophore(s) can generate radiation upon excitation due to exposure to sunlight which radiation can cause an increase in temperature of the water surrounding the pHEMA matrix. FIG. 10 depicts the experimental set-up of the method of Example 7.

### Example 8

### Adaptation of a pHEMA Matrix in Wound-Healing Therapy

The present experimental protocol aims at assessing the effect of different spectrum of light going through a pHEMA matrix on keratinocytes and on fibroblast present in a 3D-skin model and looking at the correlation between the morphometric evolution on this type of tissue and the parameters determined at the molecular level.

The pHEMA matrix used in this experiment was prepared as follows:

1) Pure water Hyclone (30 ml) #SH30529.02 from Thermo Scientific;
2) Benzoyl peroxide (100 mg) (BPO) #517909 from Sigma Aldrich Co. USA, which was used as initiator;
3) Eosin Y (50 mg) D&C Red22 #2012-27447, from Spectra Colors Corporation;
4) Fluorescein (50 mg) D&C Yellow 8 #2012-27110, from Spectra Colors Corporation;
5) 2-Hydroxyethyl methacrylate (70 ml) (HEMA) #477028, from Sigma Aldrich Co. USA; and
6) Ethylene glycol dimethacrylate (500 μl) (EGDMA) #335681, from Sigma Aldrich Co. USA, which was used as cross-linker.

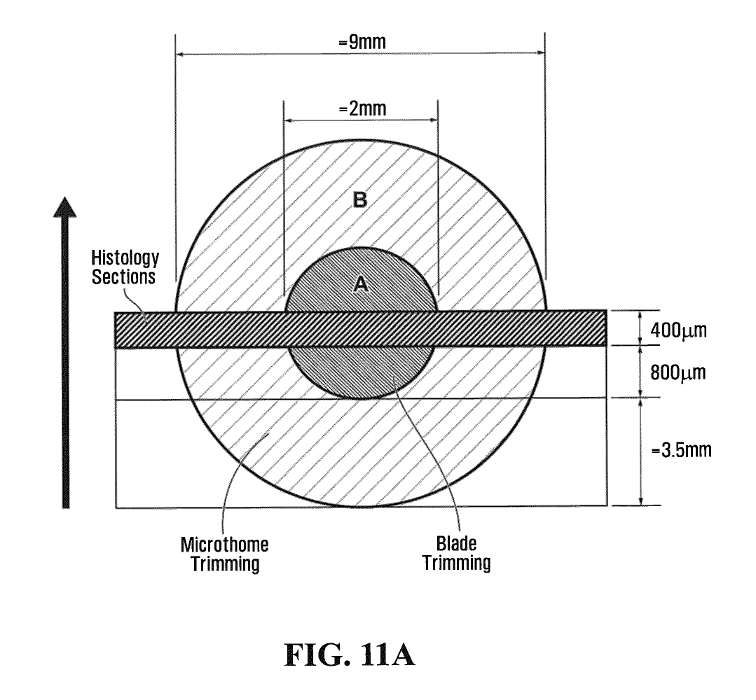

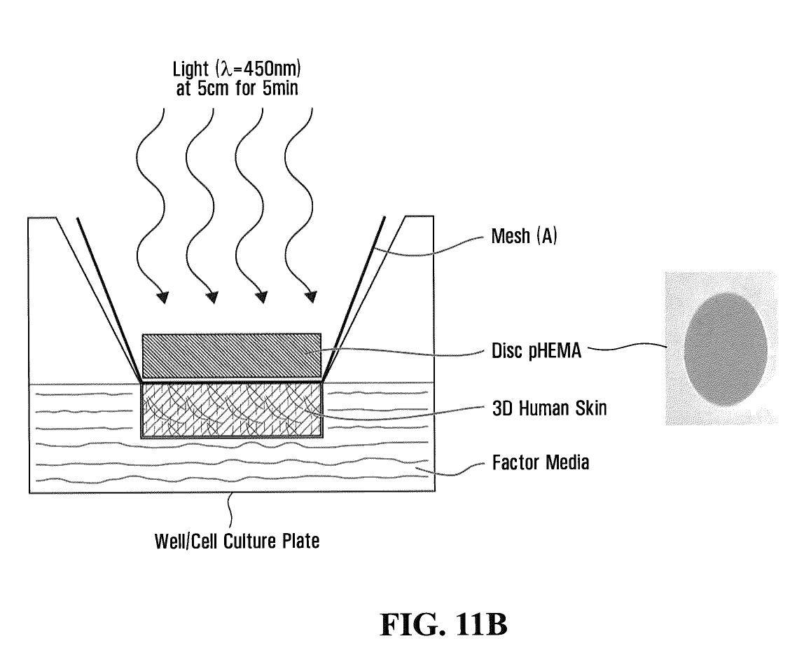

The experiment was carried out according to the experimental protocol schematized in FIGS. 11A and 11B, wherein an excision (A) was performed centrally in the skin sample so as to remove all of the layers of the skin. Histopathologic examination was performed to evaluate microscopic changes within the excised control skin and the excised treated skin. After culture (at 1 day and 3 day time points) each samples were processed and paraffin embedded (e.g., by Platform d'Histologie, Institut de recherche en immunologie et en cancérologie (I.R.I.C.) Montreal). Serial sections of 5 μm thickness were made from the paraffin blocks using a Leica microtome model RM2255 and placed on microscope slides. Table 15 below shows the light emission spectra of the pHEMA disk used in this experimental protocol.

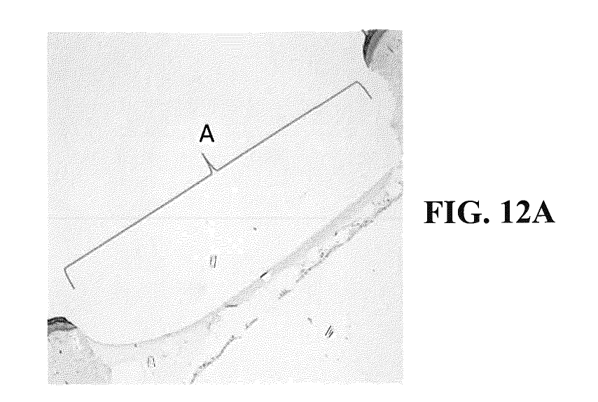

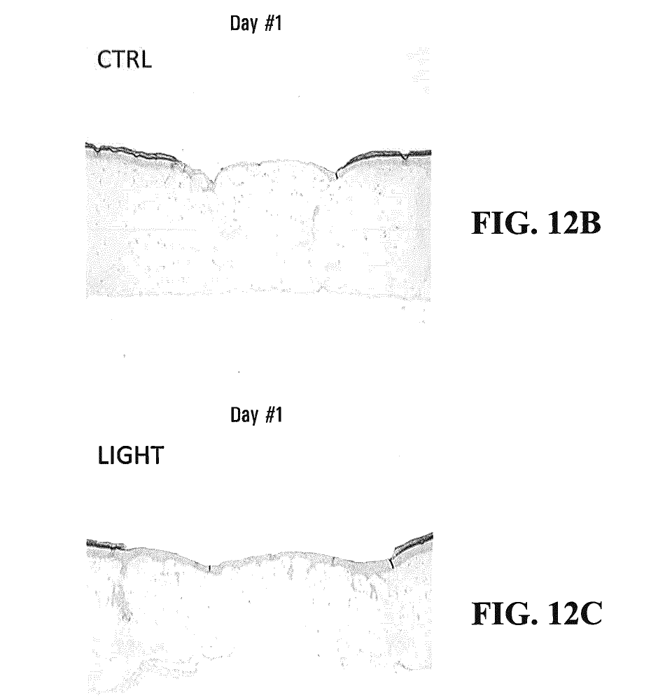

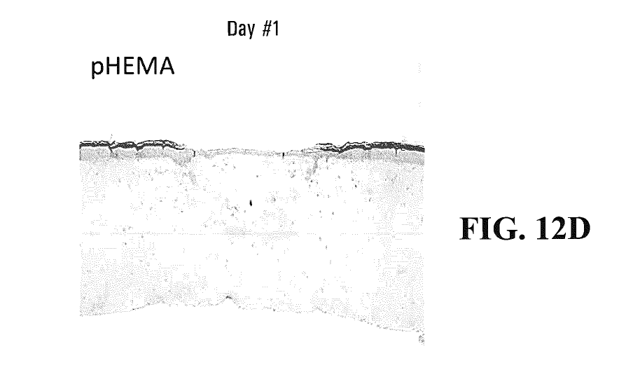

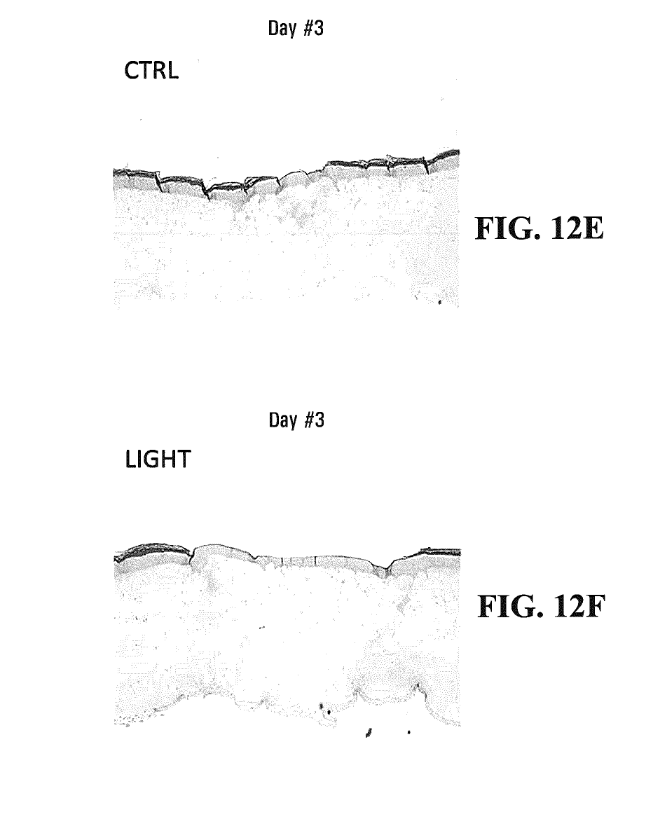

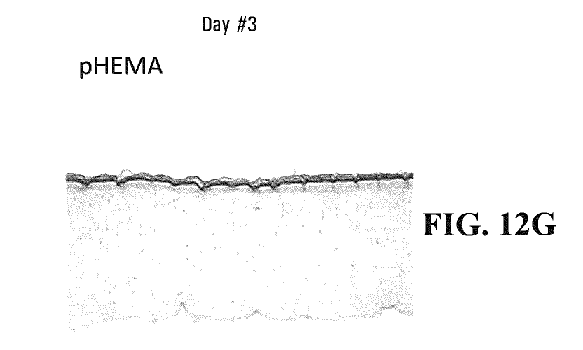

FIG. 12A is a histochemical view of full excision of 3D human skin model at day 0. FIG. 12B illustrates a histochemical view of full excision of 3D human skin model 1 day after the excision was performed wherein the skin was kept in the dark and used as control (CTRL=control). FIG. 12C is a histochemical view at 1 day following excision wherein the skin has been treated for 5 minutes with ambient light (LIGHT=ambient light). FIG. 12D is a histochemical view of full excision of 3D human skin model 1 day after the excision wherein the skin has been treated for 5 min using a 450 nm light passing through a pHEMA matrix (pHEMA)) according to the experimental protocol illustrated in FIG. 11B.

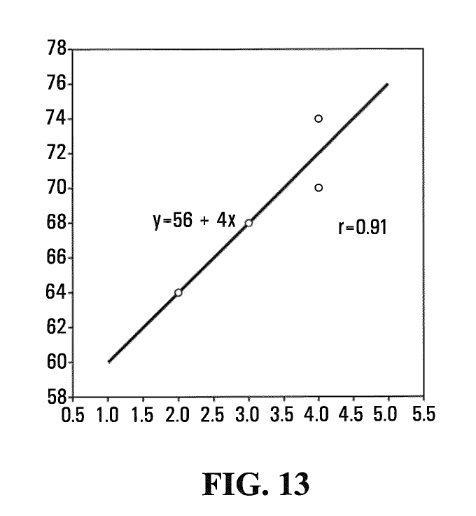

FIG. 12E is a histochemical view of full excision of 3D human skin model 3 days after the excision wherein the skin has been kept in the dark (CTRL=control). FIG. 12F is a histochemical view of full excision of 3D human skin model 3 days after the excision wherein the skin has been treated for 5 minutes with ambient light (LIGHT=ambient light). FIG. 12G is a histochemical view of full excision of 3D human skin model 3 days after the excision wherein the skin has been treated for 5 min using a 450 nm light passing through a pHEMA matrix (pHEMA)). These data show that a 5 minute treatment with a pHEMA matrix was able to close the wound created in the skin in three days. The treatment allowed regeneration of all layers of the skin and closure of the excised area. FIG. 13 indicates the relationship between the thicknesses of the epidermis versus the development of the stratum corneum 3 days post treatment with the emissive polymeric matrix of the present disclosure. Overall, these data demonstrate wound healing properties for the emissive polymeric matrices of the present disclosure.

### Example 9

### Preparation of Elastic Emissive Polymeric Matrices

The following emissive polymeric matrices were prepared according to the following protocol

1) Pure water Hyclone (25-36%) #SH30529.02 from Thermo Scientific;
2) Benzoyl peroxide (100 mg) (BPO) #517909 from Sigma Aldrich Co. USA, which was used as initiator;
3) Eosin Y (50 mg) D&C Red22 #2012-27447, from Spectra Colors Corporation;
4) Fluorescein (50 mg) D&C Yellow 8 #2012-27110, from Spectra Colors Corporation;
5) 2-Hydroxyethyl methacrylate (33-40%) (HEMA) #477028, from Sigma Aldrich Co. USA.
6) Ethylene glycol dimethacrylate (500 μl) (EGDMA) #335681, from Sigma Aldrich Co. USA, which was used as cross-linker and
7) Glycerol (18-42%) #56-81-5, from Sigma Aldrich Co. USA.

For Matrix A:

Glycerol (40%)+PHEMA (40%)+H₂O (30%), no chromophore;

For Matrix B:

Glycerol (30%)+PHEMA (40%)+H₂O (30%)+0.5 mg Eosin+0.5 mg Fluorescein;

For Matrix C:

Glycerol (18%)+PHEMA (36%)+H₂O (36%)+0.5 mg Eosin+0.5 mg Fluorescein;

For Matrix D:

Glycerol (30%)+PHEMA (40%)+H₂O (30%)+0.5 mg Eosin+0.5 mg Fluorescein;

For Matrix E:

Glycerol (36.5%)+PHEMA (36.5%)+H₂O (27%)+0.5 mg Eosin+0.5 mg Fluorescein;

For Matrix F:

Glycerol (42%)+PHEMA (33%)+H₂O (25%)+0.5 mg Eosin+0.5 mg Fluorescein

Polymerization was carried out in a nitrogen atmosphere for 2 hours at 70° C. The mixture was placed in a glass Petri dish under pressure at 76° C. for a period of between 4 and 6 hours.

### Example 10

### Elasticity of Elastic Emissive Polymeric Matrices

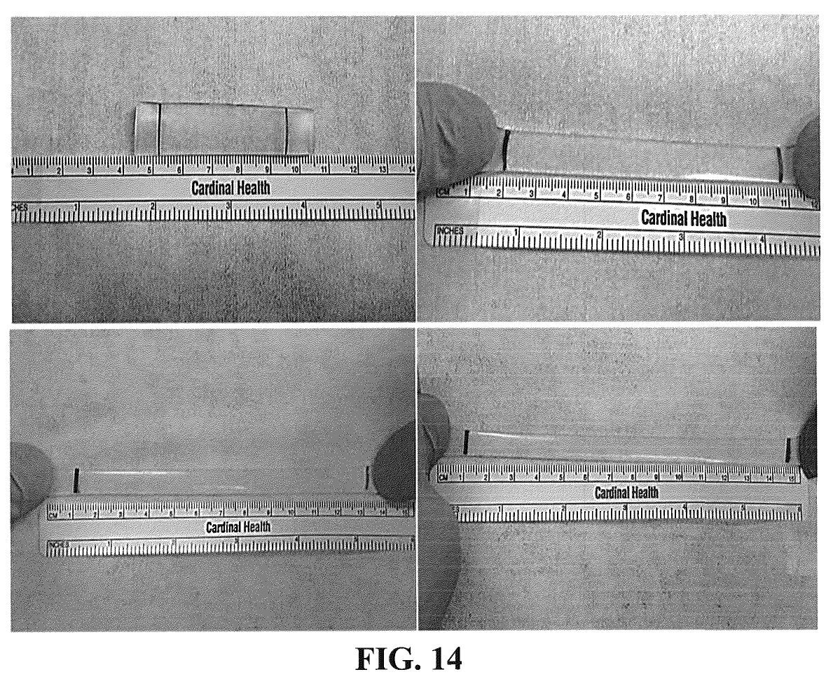

An emissive polymeric matrix was prepared according to the following protocol. The emissive polymeric matrix was prepared using the following materials: 30% glycerol, 40% pHEMA, 30% H₂O and Hydrastis canadensis as chromophore. The matrix was shaped into a rectangular piece. Marks were made on the piece at 43 mm interval such as shown on FIG. 14 and having a length of about 60 mm, a height of about 12 mm and a width of 1.20 mm (top left panel of FIG. 14). The matrix was pulled longitudinally so as to create a distance of about 87 mm between the two marks, resulting in a deformation of about 2 times the original length (top right panel of FIG. 14). The pulling forces were removed and the matrix was allowed to restore to its original shape (top left panel of FIG. 14). Again, the matrix was pulled longitudinally so as to create a distance of about 122 mm between the two marks, resulting in a deformation of about 2.8 times the original length (top right panel of FIG. 14). The pulling forces were removed and once again, the matrix was allowed to restore to its original shape (top left panel of FIG. 14). Again, the matrix was pulled longitudinally so as to create a distance of about 147 mm between the two marks, resulting in a deformation of about 3.4 times the original length (top right panel of FIG. 14). Upon removal of the pulling force, the matrix returned to its shape without showing signs of permanent deformation (data not shown), thereby demonstrating elastic properties and resilience.

### Example 11

### Leaching of Chromophores Out of Elastic Emissive Polymeric Matrices

Leaching of the chromophores out of the emissive polymeric matrix of the present disclosure was measured. The emissive polymeric matrix was prepared according to the following protocol:

1) Pure water Hyclone (25-36%) #SH30529.02 from Thermo Scientific;
2) Benzoyl peroxide (100 mg) (BPO) #517909 from Sigma Aldrich Co. USA, which was used as initiator;
3) Eosin Y (50 mg) D&C Red22 #2012-27447, from Spectra Colors Corporation;
4) Fluorescein (50 mg) D&C Yellow 8 #2012-27110, from Spectra Colors Corporation;
5) 2-Hydroxyethyl methacrylate (33-40%) (HEMA) #477028, from Sigma Aldrich Co. USA.
6) Ethylene glycol dimethacrylate (500 μl) (EGDMA) #335681, from Sigma Aldrich Co. USA, which was used as cross-linker and
7) Glycerol (18-42%) #56-81-5, from Sigma Aldrich Co. USA.

Polymerization was carried out in a nitrogen atmosphere for 2 hours at 70° C. The mixture was placed in a glass Petri dish under pressure at 76° C. for a period of between 4 and 6 hours.

The membranes as defined in Example 9 above were used for this experiment.

TABLE 24
Leaching for elastic membrane

| | Ratio | Concentration | |
|---|---|---|---|
| | Glycerol/pHEMA | of Chromophore | |
| Matrices | (v/v) | (ppm) | % Leaching |
| D (18% glycerol) | 1.00/2.00 | 5.974026 | 26.55 |
| E (30% glycerol) | 1.00/1.33 | 0.00936 | N/A |
| F (36.5% glycerol) | 1.00/1.00 | 0.684492 | 3.04 |
| G (42% glycerol) | 1.00/0.75 | 2.599694 | 11.55 |

This experiment was conducted to determine the amount of chromophore of certain emissive polymer matrices of the present disclosure. In particular, pHEMA matrices comprising Eosin Y and Fluorescein as well as the % of glycerol indicated in Table 23 above were prepared. The matrices (0.45 g each) were placed in 10 ml water for 30 days at room temperature. After this period, the water was extracted and measured on the UV-VIS at 517 nm as well as 490 nm. Measurements at these wavelengths indicated the amount of chromophore present in the water and from these values a % leaching value was determined. Based on the results, it was determined that about 6 ppm of each of the chromophore leached out of Matrix D (18% glycerol), about 2.6 ppm of each chromophore leached out of Matrix G (42% glycerol). A small but detectable amount of chromophore leached out of Matrix F (36.5% glycerol) and no detectable chromophore leached out of Matrix E (30% glycerol).

It should be appreciated that the invention is not limited to the particular embodiments described and illustrated herein but includes all modifications and variations falling within the scope of the invention as defined in the appended claims.

All documents mentioned in the specification are herein incorporated by reference.

1. An emissive polymeric matrix comprising:
2-hydroxyethyl methacrylate (HEMA), and
at least one chromophore.

2. The emissive polymeric matrix according to claim 1, being in a solid form.

3. The emissive polymeric matrix according to claim 1, being in a semi-solid form.

4. The emissive polymeric matrix according to claim 1, being elastic.

5. The emissive polymeric matrix according to claim 1, further comprising a cross-linker.

6. The emissive polymeric matrix according to claim 5, wherein the cross-linker is ethylene glycol dimethacrylate (EGDMA).

7. The emissive polymeric matrix according to claim 1, further comprising an initiator.

8. The emissive polymeric matrix according to claim 7, wherein the initiator is benzoyl peroxide.

9. (canceled)

10. The emissive polymeric matrix according to claim 1, further comprising an antimicrobial agent.

11. The emissive polymeric matrix according to claim 1, wherein the content of HEMA in the emissive polymeric matrix is between about 15 wt % and about 80 wt %.

12. (canceled)

13. The emissive polymeric matrix according to claim 1, wherein the content of HEMA in the emissive polymeric matrix is about 20 wt %.

14. The emissive polymeric matrix according to claim 1, wherein the at least one chromophore is a xanthene dye.

15. The emissive polymeric matrix according to claim 14, wherein the xanthene dye is selected from Eosin Y, Erythrosine B, Fluorescein, Rose Bengal and Phloxin B.

16. The emissive polymeric matrix according to claim 14, wherein the at least one chromophore comprises Fluorescein.

17. The emissive polymeric matrix according to claim 14, wherein the at least one chromophore comprises Eosin.

18-19. (canceled)

20. The emissive polymeric matrix according to claim 1, further comprising glycerol.

21. The emissive polymeric matrix according to claim 20, wherein the glycerol is present in an amount of between about 18 wt % and about 42 wt %.

22-25. (canceled)

26. A method for promoting wound healing comprising:
placing an emissive polymeric matrix over a wound, wherein the emissive polymeric matrix comprises 2-hydroxyethyl methacrylate (HEMA) and at least one chromophore; and
illuminating said matrix with light having a wavelength that is absorbed by the at least one chromophore;
wherein said method promotes wound healing.

27-46. (canceled)

47. A method for treatment of a skin disorder comprising:
placing an emissive polymeric matrix over a target skin tissue, wherein the emissive polymeric matrix comprises 2-hydroxyethyl methacrylate (HEMA) and at least one chromophore; and
illuminating said emissive polymeric matrix with light having a wavelength that is absorbed by the at least one chromophore; and
wherein said method promotes treatment of said skin disorder.

48-95. (canceled)

96. A kit for preparation of the emissive polymeric matrix according to claim 1, comprising the 2-hydroxyethyl methacrylate (HEMA) and the at least one chromophore and at least one container.

97-99. (canceled)
